(19) United States
(12) Patent Application Publication
(10) Pub. No.: US 2023/0324373 A1
(43) Pub. Date: Oct. 12, 2023

(54) PYRUVIC ACID OPTICAL PROBE, PREPARATION METHOD THEREFOR, AND APPLICATION THEREOF

(71) Applicant: East China University of Science and Technology, Shanghai (CN)

(72) Inventors: Yi Yang, shanghai (CN); Yuzheng Zhao, Shanghai (CN); Xie Li, Shanghai (CN); Zeyi Zhang, Shanghai (CN); Xiuze Zhang, Shanglai (CN); Li Huang, Shanghai (CN)

(73) Assignee: East China University of Science and Technology, Shanghai (CN)

(21) Appl. No.: 17/904,505

(22) PCT Filed: Feb. 9, 2021
(86) PCT No.: PCT/US2021/076317

§ 371 Date: Jun. 26, 2023

(30) Foreign Application Priority Data
Feb. 18, 2020 (CN) 202010099274.6

(51) Int. Cl. G01N 33/53; G01N 33/533; C07K 14/245

(52) U.S. Cl. CPC G01N 33/5308; G01N 33/533; C07K 14/245

(57) ABSTRACT

Disclosed in the present invention are a pyruvic acid optical probe, a preparation method therefor, and an application thereof. One aspect of the present invention is the disclosure of an optical probe, which includes a pyruvic acid-sensitive polypeptide and an optically active polypeptide, wherein the optically active polypeptide is located within the sequence of the pyruvic acid-sensitive polypeptide. The present invention also discloses a preparation method for the probe and an application of said probe in pyruvic acid measurement.

### CROSS-REFERENCE TO RELATED APPLICATIONS

This application is a U.S. National Stage Application, pursuant to 35 U.S.C. §371, of PCT International Application No. PCT/CN2021/076317, filed Feb. 9, 2021, which claims priority to and the benefit of Chinese Patent Application No. 202010099274.6, filed Feb. 18, 2020, the entire contents of each of which are incorporated by reference herein.

### TECHNICAL FIELD

The present invention relates to the field of optical sensor technology, especially to a pyruvate optical sensor, preparation method thereof and application thereof.

### SEQUENCE LISTING

This application contains a Sequence Listing which has been submitted electronically in ASCII format and is hereby incorporated by reference in its entirety. Said ASCII copy, created on Oct. 12, 2022, is named 197279_seq EN.txt, and is 102,409 bytes in size.

### BACKGROUND ART

Pyruvate is an organic acid with two functional groups, carbonyl and carboxyl, and is also the simplest α-keto acid, which is widely found in various animals and plants. Pyruvate is inseparable from energy metabolism. In the process of glycolysis, one molecule of glucose or glycogen and two molecules of ATP are consumed to produce two molecules of NADH, four molecules of ATP and two molecules of pyruvate. In the cytoplasm, pyruvate can be reduced to provide energy, and it can also be transported to the mitochondria as the main fuel of the tricarboxylic acid cycle. Pyruvate enters mitochondria under aerobic conditions, and produces NADH, CO2 and acetyl-CoA by oxidative decarboxylation catalyzed by pyruvate dehydrogenase complex. Acetyl-CoA enters the tricarboxylic acid cycle and is completely oxidized to CO2 and H₂O by oxidative phosphorylation, and the released energy can generate a large amount of ATP in the process. This is the aerobic oxidation process of sugar, and it is the main way for the body to obtain ATP.

In addition, pyruvate can be transaminated to alanine, which can be used as a raw material for protein synthesis. Pyruvate can also realize the mutual conversion of sugar, fat and amino acid in the body through acetyl-CoA and tricarboxylic acid cycle. In summary, pyruvate plays an important pivotal role in the metabolic linkages of the three major nutrients and is a key molecule critical to many aspects of eukaryotic and human metabolism.

Studies have shown that pyruvate can directly inhibit hydrogen peroxide through a non-enzymatic decarbonation reaction, and has the effect of preventing free radical damage, it has been proved to be useful in protecting the body against functional injury in cardiac reperfusion injury and acute renal failure. Pyruvate supplementation enhances the citric acid cycle. The increased production of citric acid inhibits phosphofructokinase, leading to the pentose phosphate bypass to generate reduced coenzyme II (NADPH), thereby indirectly increasing the ability of the glutathione (GSH) antioxidant system.

It is precisely because pyruvate has the above important role, the detection of pyruvate content is particularly important. Commonly used detection methods for pyruvate include UV spectrophotometry, high performance liquid chromatography, dehydrogenase colorimetry, etc. But these methods are not suitable for live cell research and have many drawbacks: they require time-consuming sample processing procedures, such as cell disruption, separation, extraction and purification; in situ, real-time, dynamic, high-throughput, and high spatiotemporal resolution detection in living cells and subcellular organelles are not possible. There remains a need in the art for methods for real-time localization, quantitative, high-throughput detection of pyruvate inside and outside cells in real time.

### SUMMARY OF INVENTION

The purpose of the present invention is to provide sensors and methods for real-time localization, high-throughput, and quantitative detection of pyruvate in and out of cells.

To achieve the above object, the invention provides the following technical solutions:

The present invention provides an pyruvate optical sensor (probe) comprising an pyruvate-sensitive polypeptide or a functional variant thereof and an optically active polypeptide or a functional variant thereof, wherein the optically active polypeptide or functional variant thereof is located within the sequence of the pyruvate-sensitive polypeptide or functional variant thereof The pyruvate-sensitive polypeptide or the functional variant thereof is divided into Part I and Part II by the optically active polypeptide or the functional variant thereof

The invention provides a pyruvate optical sensor comprising pyruvate-sensitive polypeptide B and optically active polypeptide A, wherein the optically active polypeptide A is located in the sequence of the pyruvate-sensitive polypeptide B, dividing the pyruvate-sensitive polypeptide B into the first part B1 and the second part B2, forming a sensor structure of type B1-A-B2.

In one embodiment, the pyruvate-sensitive polypeptide is a pyruvate-binding protein or the pyruvate binding domain thereof In one embodiment, the pyruvate-sensitive polypeptide is derived from E. coli. In one embodiment, the pyruvate-sensitive polypeptide is a pyruvate binding protein or a functional fragment thereof In one embodiment, the pyruvate binding protein is the PdhR protein. In one embodiment, the pyruvate-sensitive polypeptide has the sequence shown in SEQ ID NO: 1, or the sequence having at least 35%, at least 40%, at least 50%, at least 60%, at least 70%, at least 80%, at least 85%, at least 90%, at least 95%, at least 97%, at least 99% sequence identity with SEQ ID NO: 1 and retaining pyruvate-binding function. In one embodiment, the functional fragment of the pyruvate-sensitive polypeptide has a sequence of amino acids 96-254 of SEQ ID NO: 1, or a sequence having at least 35%, at least 40%, at least 50%, at least 60%, at least 70%, at least 80%, at least 85%, at least 90%, at least 95%, at least 99% identity with it and retaining pyruvate-binding function.

In one embodiment, the optically active polypeptide is a fluorescent protein or a functional variant thereof In one embodiment, the fluorescent protein is selected from the group consisting of yellow fluorescent protein (cpYFP as shown in SEQ ID No: 2), orange fluorescent protein, red fluorescent protein, green fluorescent protein (cpGFP as shown in SEQ ID No: 3), blue fluorescent protein (cpBFP as shown in SEQ ID No: 4), and apple red fluorescent protein (cpmApple as shown in SEQ ID No: 5). Preferably, the optically active polypeptide is cpYFP. In one embodiment, the fluorescent protein has a sequence as shown in any of SEQ ID NO: 2-5.

In one embodiment, the optical sensor further comprises one or more linkers flanking the optically active polypeptide. The linker of the present invention can be any amino acid sequence of any length. In one embodiment, the optically active polypeptide is flanked by a linker of no more than 5 amino acids, such as a linker of 0, 1, 2, 3, 4 amino acids. In one embodiment, the linker flanking the optically active polypeptide comprises amino acid Y. In one embodiment, linker Y is located N-terminal and/or C-terminal to the optically active polypeptide. In one embodiment, the optical sensor is shown as follows: the first part of the pyruvate-sensitive polypeptide (B1)—linker (Y)—optically active polypeptide (A)—the second part of the pyruvate-sensitive polypeptide (B2). In one embodiment, the present optical sensor does not comprise a linker.

The optically active polypeptide of the present invention can be located at any position of the pyruvate-sensitive polypeptide. In one embodiment, the optically active polypeptide is located in one or more positions of the pyruvate-sensitive polypeptide selected from the group consisting of residues 117-121, 140-143, 160-164, 174-176, 191-195 and/or 210-214, the numbering corresponding to the full length of the pyruvate binding protein. In one embodiment, the optically active polypeptide replaces one or more amino acids of the pyruvate-sensitive polypeptide at one or more positions selected from the group consisting of residues 117-121, 140-143, 160-164, 174-176, 191-195 and/or 210-214. In one embodiment, the optically active polypeptide is located in the pyruvate-sensitive polypeptide at one or more sites selected from the group consisting of: 117/118, 117/119, 117/120, 117/121, 118/119, 118/120, 118/121, 119/120, 119/121, 120/121, 140/141, 140/142, 140/ 143, 141/142, 141/143, 142/143, 160/161, 160/162, 160/163, 160/164, 161/162, 161/163, 161/164, 162/163, 162/164, 163/164, 174/175, 174/176, 175/176, 191/192, 191/193, 191/194, 191/195, 192/193, 192/194, 192/195, 193/194, 193/ 195, 194/195, 210/211, 210/212, 210/213, 210/214, 211/212, 211/213, 211/214, 212/213, 212/214 and/or 213/214. Preferably, the optically active polypeptide is located in the pyruvate-sensitive polypeptide at one or more sites selected from the group consisting of: 117/121, 141/143, 191/192, 191/193, 191/194, 191/195, 192/193, 192/194, 192/195, 193/194, 193/195, 194/195 and 210/ 214.

In one or more embodiments, the optical sensor of the present invention may be a sensor with a cpYFP inserted at one or more sites selected from the group consisting of: 117/120, 117/121, 118/119, 118/120, 118/121, 119/120, 119/121, 120/121, 140/143, 141/142, 141/143, 142/143, 160/161, 160/162, 160/ 163, 160/164, 161/163, 161/164, 162/163, 162/164, 163/164, 191/192, 191/193, 191/194, 191/195, 192/193, 192/194, 192/195, 193/194, 193/195, 194/195, 210/211, 210/212, 210/214, 211/212, 211/213, 211/214, 212/213, 212/214 of the pyruvate-binding protein or functional fragment thereof. In an exemplary embodiment, the optical sensor of the present invention may be a sensor with a cpYFP inserted in the pyruvate-binding protein or functional fragment thereof at one or more sites selected from the following: 117/121, 119/120, 119/ 121, 120/121, 140/143, 141/142, 141/143, 160/161, 160/164, 161/163, 191/192, 191/193, 191/194, 191/195, 192/193, 192/194, 192/195, 193/194, 193/195, 194/195, 210/212, 210/214, 211/213 and 211/214. Preferably, the optical sensor of the present invention can be a sensor with a cpYFP inserted in the pyruvate-binding protein or functional fragment thereof at one or more sites selected from the following: 117/121, 141/143, 191/192, 191/193, 191/194, 191/195, 192/193, 192/194, 192/195, 193/194, 193/195, 194/195 and 210/214. The functional fragment of the pyruvate-binding protein is amino acids 96-254 of SEQ ID NO: 1.

In one or more embodiments, the optical sensor of the present invention can be a sensor with a cpGFP inserted in the functional fragment of pyruvate-binding protein at one or more sites selected from the following: 117/120, 118/119, 119/ 120, 140/141, 140/142, 141/142, 141/143, 142/143, 160/161, 161/163, 161/164, 162/163, 174/175, 191/193, 210/212, 211/212, 211/214, 212/213, 212/214 and/or 213/214. In a preferred embodiment, the optical sensor of the present invention can be a sensor with a cpGFP located in the functional fragment of pyruvate-binding protein at one or more sites selected from the following: 118/119, 140/141, 160/161, 191/193, 210/212, 212/213, 212/214 and/or 213/214. The functional fragment of the pyruvate-binding protein is amino acids 96-254 of SEQ ID NO: 1.

In an exemplary embodiment, the optical sensor of the present invention may be a sensor with a cpBFP located in the pyruvate-binding protein or functional fragment thereof at one or more sites selected from the following: 117/118, 117/120, 119/120, 119/121, 140/141, 141/142, 160/162, 160/163, 161/163, 174/176, 191/192, 191/194, 192/ 193, 192/195, 193/194, 193/195, 194/195, 210/212, 211/213 or 212/213. In a preferred embodiment, the optical sensor of the present invention can be a sensor with a cpBFP located in the pyruvate-binding protein or its functional fragment at one or more sites selected from the following: 141/142, 160/163, 192/193, 192/195, 193/194, 193/195, 194/195, 210/212, 211/213. The functional fragment of the pyruvate-binding protein is positions 96-254 of SEQ ID NO: 1.

In an exemplary embodiment, the optical sensor of the present invention may be a sensor with a cpmApple located in the pyruvate-binding protein or functional fragment thereof at one or more sites selected from the following: 117/119, 117/120, 118/119, 118/120, 119/121, 140/141, 140/142, 141/142, 160/161, 160/162, 160/164, 162/163, 174/ 176, 175/176, 191/194, 191/195, 192/193, 192/194, 192/195, 193/194, 210/212, 211/213, 211/214, 212/214. In a preferred embodiment, the optical sensor of the present invention can be a sensor with a cpmApple located in the pyruvate-binding protein or its functional fragment at one or more sites of selected from the following: 117/120, 118/120, 160/162, 162/163, 191/195, 192/193, 192/194, 192/195, 210/212, 211/213. The functional fragment of the pyruvate-binding protein is positions 96-254 of SEQ ID NO: 1.

In one embodiment, the optical sensors of the present invention have or consist of the sequences shown in SEQ ID NO: 6-18.

The present invention also provides mutants of pyruvate-sensitive polypeptides. The mutation is located at 1, 2, 3, 4, 5, 6 or 7 sites selected from the group consisting of: Q138, S190, R191, R192, E193, M194, and L195 of the pyruvate-binding protein or its functional fragment. Illustratively, the mutations are selected from 1, 2, 3, 4, 5, 6 or 7 mutations of the following: Q138S, Q138Y, Q138C, Q138L, Q138P, Q138H, Q138R, Q138W, Q138I, Q138T, Q138N, Q138K, Q138F, Q138V, Q138A, Q138D, Q138E, Q138M, Q138A, S190E, R191S, R191Y, R191C, R191L, R191P, R191H, R191Q, R191W, R191I, R191T, R191N, R191K, R191F, R191V, R191A, R191D, R191E, R191M, R191A, R192D, E193S, E193Y, E193C, E193L, E193P, E193H, E193Q, E193W, E193R, E193I, E193T, E193N, E193K, E193M, E193V, E193F, E193D, E193A, E193G, M194S, M194Y, M194C, M194L, M194P, M194H, M194Q, M194W, M194R, M1941, M194A, M194N, M194K, M194T, M194V, M194F, M194D, M194E, T102G, L195S, L195Y, L195C, L195D, L195P, L195H, L195Q, L195W, L195I, L195T, L195N, L195K, L195R, L195V, L195A, L195F, L195E, L195M and/or L195A.

The present invention also provides optical sensors comprising pyruvate-sensitive polypeptides with one or more mutations. In one or more embodiments, the optical sensor is any optical sensor inserted with an optically active polypeptide as described above, and the pyruvate-sensitive polypeptide in the optical sensor has mutations at 1, 2, 3, 4, 5, 6 or 7 sites selected from the group consisting of: Q138, S190, R191, R192, E193, M194, and L195. In one or more embodiments, the optical sensor comprising a mutated pyruvate-sensitive polypeptide shows no less response to pyruvate than its non-mutated counterpart. In one or more embodiment, the mutations are selected from 1, 2, 3, 4, 5, 6 or 7 of the following: Q138S, Q138Y, Q138C, Q138L, Q138P, Q138H, Q138R, Q138W, Q1381, Q138T, Q138N, Q138K, Q138F, Q138V, Q138A, Q138D, Q138E, Q138M, Q138A, S190E, R191S, R191Y, R191C, R191L, R191P, R191H, R191Q, R191W, R191I, R191T, R191N, R191K, R191F, R191V, R191A, R191D, R191E, R191M, R191A, R192D, E193S, E193Y, E193C, E193L, E193P, E193H, E193Q, E193W, E193R, E193I, E193T, E193N, E193K, E193M, E193V, E193F, E193D, E193A, E193G, M194S, M194Y, M194C, M194L, M194P, M194H, M194Q, M194W, M194R, M1941, M194A, M194N, M194K, M194T, M194V, M194F, M194D, M194E, T102G, L195S, L195Y, L195C, L195D, L195P, L195H, L195Q, L195W, L195I, L195T, L195N, L195K, L195R, L195V, L195A, L195F, L195E, L195M and/or L195A.

In an exemplary embodiment, the B 1-A-B2 optical sensor of the present invention may be a sensor with a cpYFP inserted into a functional fragment of the pyruvate-binding protein at the position of 141/143, 191/193, 192/194 or 192/195, and has mutations of Q138P, Q138L, R191Y, R191F, R191L, R191P, E193Q, E193L, M194D, M194V, M194H, M194W, M194V/S190E/R191N/R192D, M194V/S190D/R191Y/R192T, S 190P/R191H/R192P, S 190R/R191S/R192P, S190L/R191V, S190T/R191Q/R192E and/or R191S/R192T, the numbering corresponding to the full length of the pyruvate binding protein. In one or more embodiments, the optical sensors of the present invention are mutants based on sensors with different insertion sites, wherein the combination of insertion sites and mutations is selected from: 191/193-Q138P, 191/193-Q138L, 191 /193-R191Y, 191/193-R191F, 191/193-R191L, 191/193-R191P, 191/193-E193Q, 191/193-E193L, 192/194-M194D, 192/194-M194V, 192/194 -M194H, 192/194-M194W, 192/194-M194V-S 190E/R191N/R192D, 192/194-M194V/S190D/R191Y/R192T, 141/143-S190P/R191H/R192P, 141/143-S190R/R /R192P, 141/143-S190L/R191V, 141/143-S190T/R191Q/R192E and 141/143-R191S/R192T. In an exemplary embodiment, the pyruvate-binding protein functional fragment is amino acids 96-254 of SEQ ID NO: 1. Preferably, the optical sensors of the present invention have or consist of the sequences shown in SEQ ID NO: 19-30.

The optical sensors of the present invention comprise any of the amino acid sequence of SEQ ID NO: 6-30 or a variant thereof In one embodiment, the optical sensor provided by the present invention comprises a sequence with 35%, 40%, 50%, 60%, 70%, 80%, 85%, 90%, 95%, 99% identity to any of the amino acid sequences SEQ ID NO: 6-30. In a preferred embodiment, the optical sensor provided by the present invention comprises a sequence substantially similar or identical to any of the amino acid sequences SEQ ID NO: 6-30. In a more preferred embodiment, the optical sensor provided by the present invention comprises or consists of any one of SEQ ID NO: 19-30.

The invention also provides fusion polypeptides comprising the optical sensors described herein and other polypeptides. In some embodiments, other polypeptides are located N- and/or C-terminal to the said optical sensor. In some embodiments, other polypeptides include polypeptides that localize optical sensors to different organelles or subcellular organelles, tags for purification, or tags for immunoblotting.

The invention also provides nucleic acid sequences encoding the polypeptides, sensors or proteins described herein, or complements thereof In one embodiment, the nucleic acid sequence of the present invention is selected from (1) the coding sequence of the amino acid sequence shown in any of SEQ ID NOs: 6-30 or its complement, (2) and (1) having at least 99%, Sequences that are at least 95%, at least 90%, at least 80%, at least 70% or at least 50% identical, fragments of (3) (1) or (2). In one or more embodiments, the fragments are primers. In one embodiment, the nucleic acid sequence of the invention comprises SEQ ID No: 31 or a variant or fragment thereof In a preferred embodiment, the present invention provides a nucleic acid sequence comprising at least 99%, at least 95%, at least 90%, at least 80%, at least 70% or at least 50% identical to the nucleotide sequence SEQ ID NO: 31. The present invention also relates to complementary sequences of the above nucleic acid sequences or variants thereof, which may comprise nucleic acid sequences or complementary sequences thereof encoding fragments, analogs, derivatives, soluble fragments and variants of the optical sensors or fusion polypeptides of the present invention.

The invention also provides nucleic acid constructs comprising the nucleic acid sequences described herein, or their complementary sequences, which encode the optical sensors or fusion polypeptides described herein. In one or more embodiments, the nucleic acid construct is a cloning vector, expression vector, or recombinant vector. In one or more embodiments, the nucleic acid sequence is operably linked to an expression control sequence. In some embodiments, the expression vector is selected from the group consisting of prokaryotic expression vector, eukaryotic expression vector, and viral vector.

The invention also provides cells comprising the nucleic acid sequences or nucleic acid constructs of the invention. In one or more embodiments, the cells express an optical sensor or fusion polypeptide described herein. The present invention also provides a detection kit comprising a pyruvate optical sensor or fusion polypeptide described herein or a pyruvate optical sensor or fusion polypeptide prepared as described herein.

The invention provides methods for preparing the optical sensors described herein, comprising providing cells expressing the optical sensors or fusion polypeptides described herein, culturing the cells under conditions in which the cells express them, and isolating the optical sensors or fusion polypeptides. In one embodiment, a method of preparing a pyruvate optical sensor or fusion polypeptide described herein comprises: 1) transferring an expression vector encoding a pyruvate optical sensor described herein into a host cell; 2) culturing the host cell under conditions suitable for the expression of the expression vector, and 3) isolating the pyruvate optical sensor.

The invention also provides a method for detecting pyruvate in a sample, comprising contacting the sample with an optical sensor or fusion polypeptide described herein, or an optical sensor or fusion polypeptide prepared according to the method described herein, and detecting changes in an optically active polypeptide. The detecting may be performed in vivo, in vitro, subcellularly, or in situ. Said sample is such as blood.

The invention also provides a method for quantitating pyruvate in a sample, comprising contacting the sample with an optical sensor or fusion polypeptide described herein or an optical sensor or fusion polypeptide prepared according to the method described herein, detecting changes in optically active polypeptide, and quantitating pyruvate in the sample according to the changes in optically active polypeptide.

The invention also provides a method for screening compounds, such as pharmaceuticals, comprising contacting a candidate compound with an optical sensor or fusion polypeptide described herein, or an optical sensor or fusion polypeptide prepared as described herein, detecting changes in the optically active polypeptide, and screening the compounds according to the changes in the optically active polypeptide. Said method allows high-throughput screening of compounds.

The invention also provides use of a pyruvate optical sensor or fusion polypeptide described herein or a pyruvate optical sensor or fusion polypeptide prepared according to the methods described herein in intracellular/extracellular localization of pyruvate. In one or more embodiments, the localization is in real time.

Beneficial effects of the present invention: The pyruvate optical sensor provided by the invention is easy to mature, has large dynamic change of fluorescence and good specificity and it can be expressed in cells through the method of gene manipulation, and it can locate, high-throughput, and quantitatively detect pyruvate in and out of cells in real time, eliminating the time-consuming steps of sample processing. The experimental results show that the highest response of the pyruvate optical sensor provided in this application to pyruvate is more than 10 times that of the control, and it can locate, qualitatively and quantitatively detect cells in subcellular structures such as cytoplasm, mitochondria, nucleus, endoplasmic reticulum, lysosome and Golgi apparatus, and can perform high-throughput compound screening and quantitative detection of pyruvate in blood.

### BRIEF DESCRIPTION OF THE DRAWINGS

The invention is further illustrated below in combination with the drawings and examples.

FIG. 1 is an SDS-PAGE graph of the exemplary pyruvate optical sensor as described in Example 1;

FIG. 2 is a graph of the response change of the exemplary pyruvate optical sensor to pyruvate as described in Example 2, the sensor comprises cpYFP and pyruvate-binding protein;

FIG. 3 is a graph of the response change of the exemplary pyruvate optical sensor to pyruvate as described in Example 3, the sensor comprises cpGFP and pyruvate-binding protein;

FIG. 4 is a graph of the response change of the exemplary pyruvate optical sensor to pyruvate as described in Example 4, the sensor comprises cpBFP and pyruvate-binding protein;

FIG. 5 is a graph of the response change of the exemplary pyruvate optical sensor to pyruvate as described in Example 5, the sensor comprises cpmApple and pyruvate-binding protein;

FIG. 6 is the response to pyruvate of an exemplary pyruvate optical sensor having cpYFP inserted at sites 191/193, 192/194 or 192/195 of the pyruvate-binding protein and containing mutations as described in Example 6;

FIGS. 7A-7B are titration curves of the exemplary pyruvate optical sensor to different concentrations of pyruvate described in Example 7;

FIGS. 8A-8C are graphs of the fluorescence spectral properties of the exemplary pyruvate optical sensor described in Example 8;

FIG. 9 is a bar graph of the detection for specificity of the exemplary pyruvate optical sensor described in Example 8;

FIG. 10 is a photograph of the subcellular organelle localization of the exemplary pyruvate optical sensor in mammalian cells as described in Example 9;

FIG. 11 is a schematic diagram of the response of an exemplary pyruvate optical sensor to exogenous pyruvate in mammalian cells as described in Example 10;

FIG. 12 is a dot plot of the exemplary pyruvate optical sensor for high-throughput compound screening in living cells as described in Example 11;

FIG. 13 is a bar graph of the quantification of pyruvate in mouse and human blood by the exemplary pyruvate optical sensor as described in Example 12.

### DETAILED DESCRIPTION

When a value or range is given, the term “about” used herein means that the value or range is within 20%, within 10%, and within 5% of the given value or range.

The term “pyruvate-sensitive polypeptide” or “pyruvate-responsive polypeptide” used herein refers to a polypeptide which responds to pyruvate production including any response in chemical, biological, electrical, or physiological parameters of a polypeptide that interacts with the sensitive-polypeptide. The response includes small changes, for example, changes in the orientation of the amino acid or peptide fragment of the polypeptide and, for example, changes in the primary, secondary or tertiary structure of the polypeptide, including, for example, changes in protonation, electrochemical potential and/or conformation. “Conformation” is the three-dimensional arrangement of the primary, secondary, and tertiary structure of a molecule containing side groups in the molecule; when the three-dimensional structure of the molecule changes, the conformation changes. Examples of conformational changes include transitions from α-helix to β-fold or from β-fold to α-helix. It should be understood that detectable alterations need not be conformational changes as long as the fluorescence of the fluorescent protein moiety is altered. The pyruvate-sensitive polypeptide described herein may also include functional variants thereof. The functional variants of the pyruvate-sensitive polypeptide include, but are not limited to, variants that can interact with pyruvate and thereby undergo the same or similar changes as the parent pyruvate-sensitive polypeptide.

The pyruvate-sensitive polypeptides of the present invention include, but are not limited to, the pyruvate-binding protein PdhR or its variants with more than 90% homology. The exemplary pyruvate-binding protein PdhR described in the present invention is derived from Escherichia coli. PdhR is a bacterial transcription factor consisting of a pyruvate-binding/regulatory domain and a DNA-binding domain. An exemplary PdhR protein is shown in SEQ ID NO:1. In one or more embodiments, the pyruvate-sensitive polypeptide comprises the pyruvate-binding domain of the PdhR protein, i.e., amino acids 96-254, but does not include the DNA-binding domain. When describing an optical sensor or pyruvate-binding protein of the invention (e.g., when describing an insertion site or a mutation site), references to amino acid residue numbering refer to SEQ ID NO: 1.

The term “optical sensor” as used herein refers to a pyruvate-sensitive polypeptide fused to an optically active polypeptide. The inventors discovered that the conformational changes produced by binding an pyruvate-sensitive polypeptide, such as an pyruvate binding protein to a physiological concentration of pyruvate specifically by pyruvate sensitive polypeptides causes conformational changes in an optically active polypeptides, such as fluorescent proteins, which in turn result in the alteration of the optical properties of the optically active polypeptides. A standard curve is plotted with the help of the fluorescence of fluorescent proteins determined at different concentrations of pyruvate allows the presence and/or level of pyruvate to be detected and analyzed.

In the present optical sensor, an optically active polypeptide, such as a fluorescent protein, is operably inserted into a pyruvate-sensitive polypeptide. The protein-based “optically active polypeptides” are polypeptides with the ability to emit fluorescence. In the present optical sensor, an optically active polypeptide, such as a fluorescent protein, is operably inserted into a pyruvate-sensitive polypeptide. Preferably, the protein substrate is selected to have fluorescence properties that are easily distinguishable in the unactivated and activated conformational states. The optically active polypeptide described herein may also include a functional variant thereof A functional variant of the optically active polypeptide includes, but is not limited to, a variant for which the same or similar change in fluorescence property may occur as the parent optically active polypeptide.

A “linker” or “linker region” refers to an amino acid or nucleotide sequence linking two parts in a polypeptide, protein, or nucleic acid of the invention. Exemplarily, the number of amino acids at the amino terminus of the linker region of the pyruvate-sensitive polypeptide to the optically active polypeptide in the invention is selected to be 0-3, and the number of amino acids at the carboxyl terminus is selected to be 0-2; when a recombinant optical sensor is linked to a functional protein as a basic unit, it can be fused at the amino or carboxyl terminus of the recombinant optical sensor. The linker sequence may be a short peptide chain composed of one or more flexible amino acids, as in Y.

The term “fluorescent protein” used herein refers to a protein that fluoresces upon irradiation with excitation light. For example, green fluorescent protein GFP and circularly permuted blue fluorescent protein (cpBFP), circularly permuted green fluorescent protein (cpGFP), circularly permuted yellow fluorescent protein (cpYFP), etc.; there are also red fluorescent protein RFP commonly used in the art, and cyclic permuted proteins derived from this protein, such as cpmApple, cpmOrange, cpmKate and the like. Those skilled in the art know fluorescent proteins and sequences thereof that can be used for the invention. Exemplarily, cpYFP is shown in SEQ ID No: 2; cpGFP is shown in SEQ ID No: 3; cpBFP is shown in SEQ ID No: 4; and cpmApple is shown in SEQ ID No: 5.

The pyruvate optical sensor of the present invention includes amino acids 96-254 of the pyruvate-binding domain of a pyruvate-sensitive polypeptide (B), such as a pyruvate-binding protein or a variant thereof, and an optically active polypeptide (A), such as a fluorescent protein. The optically active polypeptide (A) is inserted into amino acids 96-254 of the pyruvate-binding domain of the pyruvate-sensitive polypeptide (B), and B is divided into two parts, B1 and B2, to form a sensor structure of the formula B 1-A-B2; the interaction of pyruvate-sensitive polypeptide B and pyruvate results in an enhanced optical signal of the optically active polypeptide (A).

In the present optical sensor, the optically active polypeptide may be located anywhere in the pyruvate-sensitive polypeptide. In one embodiment, the optically active polypeptide is located anywhere in N-C direction in a pyruvate-sensitive polypeptide in N-C direction. Specifically, the optically active polypeptide is located in a flexible region of a pyruvate-sensitive polypeptide that refers to some specific structures present in higher structures of a protein such as loop domains, which are more mobile and flexible than other higher structures of the protein, and this region may undergo dynamic changes in spatial structural conformation upon binding of this protein and a ligand. The flexible region mentioned in the present invention mainly refers to the region where the insertion site is located in the pyruvate-binding protein, such as the regions of amino acid residues 117-121, 140-143, 160-164, 174-176, 191-195 and 210-214. Exemplarily, the optically active polypeptide is located in the amino acid sequence of the pyruvate-binding protein at one or more positions selected from the group consisting of: 117/118, 117/119, 117/120, 117/121, 118/119, 118/120, 118/121, 119/120, 119/121, 120/121, 140/141, 140/142, 140/143, 141/142, 141/143, 142/143, 160/161, 160/162, 160/163, 160/164, 161/162, 161/163, 161/164, 162/163, 162/164, 163/164, 174/175, 174/176, 175/176, 191/192, 191/193, 191/194, 191/195, 192/193, 192/194, 192/195, 193/194, 193/195, 194/195, 210/211, 210/212, 210/213, 210/214, 211/212, 211/213, 211/214, 212/213, 212/214 or 213/214. In a preferred embodiment, the optically active polypeptide is located in the amino acid sequence of the pyruvate-binding protein or functional fragment thereof at one or more positions selected from the group consisting of: 117/121, 141/143, 191/192, 191/193, 191/194, 191/195, 192/193, 192/194, 192/195, 193/194, 193/195, 194/195 or 210/214. In the present invention, if the two numbers in a site represented in “X/Y” form are consecutive integers, this means that the optically active polypeptide is located between the amino acids stated by those numbers. For example, insertion site 117/118 indicates that the optically active polypeptide is located between amino acids 117 and 118 of the pyruvate-sensitive polypeptide. If the two numbers in a site represented in “X/Y” form are not consecutive integers, it indicates that the optically active polypeptide displaces the amino acids between the amino acids indicated by those numbers. For example, insertion site 191/195 indicates the replacement of amino acids 192-194 of the lactate-sensitive polypeptide by an optically active polypeptide. In a preferred embodiment, the optically active polypeptide is located in the amino acid sequence of the pyruvate-binding protein or functional fragment thereof at one or more positions selected from the group consisting of: 117/118, 117/119, 117/120, 117/121, 118 /119, 118/120, 118/121, 119/120, 119/121, 120/121, 140/141, 140/142, 140/143, 141/142, 141/143, 142/143, 160/161, 160/162, 160/163, 160/164, 161/162, 161/163, 161/164, 162/163, 162/164, 163/164, 174/175, 174/176, 175/176, 191 /192, 191/193, 191/194, 191/195, 192/193, 192/194, 192/195, 193/194, 193/195, 194/195, 210/211, 210/212, 210/213 , 210/214, 211/212, 211/213, 211/214, 212/213, 212/214 or 213/214, the functional fragment comprises amino acids 96-254 of the pyruvate-binding protein. In a more preferred embodiment, the optically active polypeptide is located in the amino acid sequence of the pyruvate-binding protein functional fragment comprising amino acids 96-254 at one or more positions selected from the group consisting of: 117/121, 141/143, 191/192, 191/193, 191/194, 191/195, 192/193, 192/194, 192/195, 193/194, 193/195, 194/195 or 210/ 214, as shown in SEQ ID NO: 6-18.

When referring to a certain polypeptide or protein, the term “variant” or “mutant” used herein includes variants that have the same function of said polypeptide or protein but differ in sequence. Variants of polypeptides or proteins can include: homologous sequences, conservative variants, allelic variants, natural mutants, induced mutants. These variants include, but are not limited to: deletions, insertions and/or substitutions of one or more (usually 1-30, preferably 1-20, more preferably 1-10, most preferably 1-5) amino acids in the sequence of the polypeptide or protein, and sequences obtained by adding one or more (usually within 20, preferably within 10, more preferably within 5) amino acids to its carboxy terminus and/or amino terminus. These variants may also comprise at least about 70%, at least about 75%, at least about 80%, at least about 85%, at least about 90%, at least about 95%, at least about 98%, at least about 99% or 100% sequence identity to the polypeptide or protein. Not limited by theory, an amino acid residue is altered without changing the overall configuration and function of the polypeptide or protein, i.e., a functionally conserved mutation. For example, in the art, substitution with amino acids of approaching or similar perperty generally does not alter the function of a polypeptide or protein. In the art, amino acids with similar property tend to refer to families of amino acids with similar side chains, which have been well defined in the art. These families include amino acids with basic side chain (e.g., lysine, arginine, histidine), amino acids with acidic side chain (e.g., aspartic acid, glutamic acid), amino acids with uncharged polar side chain (e.g., glycine, asparagine, glutamine, serine, threonine, tyrosine, cysteine), amino acids with non-polar side chain (e.g., alanine, valine, leucine, isoleucine, arginine, phenylalanine, methionine, pyruvate), amino acids with β-branched side chain (e.g., threonine, valine, isoleucine) and amino acids with aromatic side chain (e.g., tyrosine, phenylalanine, tryptophan, histidine). For another example, the addition of one or more amino acids to the amino terminus and/or carboxy terminus also generally does not alter the function of a polypeptide or protein. The conservative amino acid substitutions of many commonly known non-genetic coding amino acids are known in the art. Conservative substitutions of other non-coding amino acids can be determined based on a comparison of their physical properties with those of the amino acids that are genetically encoded.

In two or more sequences of a polypeptide or nucleic acid molecule, the terms “identity” or “percent identity” refer to when the maximum correspondence is compared and aligned by manual alignment and visual inspection using methods known in the art such as sequence comparison algorithms, in a comparison window or a specified region, two or more sequences or subsequences are identical or have a certain percentage of amino acid residues or nucleotides that are identical in the specified region (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 100% identical). For example, the preferred algorithms suitable for determination of percent identity and percent similarity are the BLAST and BLAST 2.0 algorithms, respectively, see Altschul et al., (1977) nucleic acids res. 25:3389 and Altschul et al., (1990) J. mol. Biol 215:403.

Those skilled in the art is well known that in gene cloning operation, it is often necessary to design a suitable restriction enzyme cutting site, which is bound to introduce one or more incoherent residues at the ends of the expressed polypeptide or protein, and this does not affect the activity of the polypeptide or protein of interest. Also for constructing fusion proteins, facilitating the expression of recombinant proteins, obtaining recombinant proteins that are automatically secreted outside the host cell, or facilitating the purification of recombinant proteins, it is often necessary to add some amino acids to the N-terminus, C-terminus, or other suitable regions within the recombinant protein, for example, including but not limited to, suitable linker peptides, signal peptides, leader peptides, terminal extensions, glutathione S-transferases (GSTs) maltose E-binding protein, Protein A, tags such as 6His or flag, or the proteolytic enzyme sites of Factor Xa or thrombin or enterokinase.

The present optical sensor may comprise a pyruvate-sensitive polypeptide with a mutation. Such mutations are, for example, mutations at Q138, S190, R191, R192, E193, M194, and/or L195. Exemplarily, the mutation is selected from one or more of the following: Q138S, Q138Y, Q138C, Q138L, Q138P, Q138H, Q138R, Q138W, Q1381, Q138T, Q138N, Q138K, Q138F, Q138V, Q138A, Q138D, Q138E, Q138M, Q138A, S190E, R191S, R191Y, R191C, R191L, R191P, R191H, R191Q, R191W, R191I, R191T, R191N, R191K, R191F, R191V, R191A, R191D, R191E, R191M, R191A, R192D, E193S, E193Y, E193C, E193L, E193P, E193H, E193Q, E193W, E193R, E193I, E193T, E193N, E193K, E193M, E193V, E193F, E193D, E193A, E193G, M194S, M194Y, M194C, M194L, M194P, M194H, M194Q, M194W, M194R, M194I, M194A, M194N, M194K, M194T, M194V, M194F, M194D, M194E, T102G, L195S, L195Y, L195C, L195D, L195P, L195H, L195Q, L195W, L1951, L195T, L195N, L195K, L195R, L195V, L195A, L195F, L195E, L195M and/or L195A. In preferred embodiments, the mutation is selected from one or more of the following: Q138P, Q138L, S190E, R191Y, R191N, R191F, R191L, R191P, R192D, E193Q, E193L, M194D, M194V, M194H, M194W, M194V and S190E and R191N and R192D (M194V/S190E/R191N/R192D), M194V and S190D and R191Y and R192T (M194V/S190D/R191Y/R192T), S190P and R191H and R192P (S190P/R191H) S190R and R191S and R192P (S190R/R191S/R192P), S190L and R191V (S190L/R191V), S190T and R191Q and R192E (S190T/R191Q/R192E) or R191S and R192T (R191S/R192T).

In an exemplary embodiment, the B 1-A-B2 optical sensor of the present invention may be a sensor with a cpYFP inserted at one or more sites selected from 117/121, 141/143, 191/192 of PdhR(96-254) having one or more mutations selected from the group consisting of: Q138P, Q138L, R191Y, R191F, R191L, R191P, E193Q, E193L, M194D, M194V, M194H, M194W, M194V and S190E and R191N and R192D (M194V/S190E/R191N/R192D), M194V and S190D and R191Y and R192T (M194V/S190D/R191Y/R192T), S190P and R191H and R192P (S190P/R191H/R192P), S190R and R191S and R192P (S192P), S1991S/R192P (S192P) and R191V (S190L/R191V), S190T and R191Q and R192E (S190T/R191Q/R192E) or R191S and R192T (R191S/R192T). Preferably, the optical sensor is a sensor in which cpYFP is inserted into the 191/193 position of PdhR (96-254) having a mutation of Q138P, Q138L, R191Y, R191F, R191L, R191P, E193Q or E193L, or a sensor with cpYFP inserted at positions 192/194 of PdhR(96-254) having a mutation of M194D, M194V, M194H or M194W. In one embodiment, the combination of the insertion site of the optically active polypeptide in the pyruvate-sensitive polypeptide and the mutation of the pyruvate-sensitive polypeptide is selected from one or more of the following: 191/193-Q138P, 191/193-Q138L, 191/193-R191Y, 191/193-R191F, 191/193-R191L, 191/193-R191P, 191/193-E193Q, 191/193-E193L, 192/194-M194D, 192/194-M194V, 192/194-M194H, 192/194-M194W 192/194-M194V/S190E/R191N/R192D, 192/194-M194V/S190D/R191Y/R192T, 141/143-S190P/R191H/R192P, 141/143-S190R/R191S/R192P, 141/143-S190L/R191V, 141/143-S190T/R191Q/R192E and/or 141/143-R191S/R192T. The sequences of these optical sensors are shown in SEQ ID NO: 19-30.

The terms “functional fragment”, “derivative” and “analog” as used herein refer to a protein that retains substantially the same biological function or activity as the original polypeptide or protein (eg, PdhR protein or fluorescent protein). Functional variants, derivatives, or analogues of a polypeptide or protein (e.g., a PdhR protein or a fluorescent protein) of the invention may be (i) a protein having one or more conservative or nonconservative amino acid residues (preferably conservative amino acid residues) substituted, whereas such substituted amino acid residues may or may not be encoded by the genetic code, or (ii) a protein having a substituted group in one or more amino acid residues, or (iii) a protein formed by the fusion of the mature protein to another compound, such as a compound that extends the half-life of the protein, such as polyethylene glycol, or (iv) a protein formed by the fusion of an additional amino acid sequence to this protein sequence (such as a secretory sequence or the sequence or protein used to purify this protein, or the fusion polypeptide formed with an antigenic IgG fragment). According to the teaching herein, these functional variants, derivatives, and analogues belong to the common knowledge to those skilled in the art. The difference of said analogues from the original polypeptide or protein may be a difference in amino acid sequence, a difference in modified form that does not affect the sequence, or both. These proteins include natural or induced genetic variants. Induced variants can be derived by various techniques, such as random mutagenesis by radiation or exposure to mutagens and can also be obtained by site directed mutagenesis or other known techniques in molecular biology.

The present fusion polypeptide comprises the optical sensor described herein and an additional polypeptide. In some embodiments, the optical sensor described herein also comprises another polypeptide fused to it. The additional polypeptide described herein does not affect the property of the optical sensor. The additional polypeptide may be located N- and/or C-terminal to the optical sensor. In some embodiments, other polypeptides include polypeptides that localize optical sensors to different organelles or subcellular organelles, tags for purification, or tags for immunoblotting. There may be a linker between the optical sensor and the additional polypeptide in the fusion polypeptide described herein. The subcellular organelles described here include cytosol, mitochondria, nucleus, endoplasmic reticulum, cell membrane, Golgi apparatus, lysosome, and peroxisome, among others. In some embodiments, the tag for purification or the tag for immunoblotting include 6 histidine (6*His), glutathione sulfurtransferase (GST), Flag.

The expression vector of the present invention comprises the nucleic acid sequence of the present invention or a complementary sequence thereof operably linked to an expression control sequence, the nucleic acid sequence encoding the optical sensor or fusion polypeptide of the present invention. In some embodiments, the expression vector is selected from the group consisting of prokaryotic expression vector, eukaryotic expression vector, and viral vector. In some embodiments, prokaryotic expression vectors are preferably derived from plasmid pCDFDuet-1 operably linked to the nucleic acid sequences described herein. In some embodiments, expression control sequences include origins of replication, promoters, enhancers, operators, terminators, ribosome binding sites.

The present invention also provides a method for preparing the above-mentioned pyruvate optical sensor, comprising the following steps: 1) incorporating the nucleic acid sequence encoding the pyruvate optical sensor described herein into an expression vector; 2) transferring the expression vector into the host cell; 2) culturing the host cell under conditions suitable for the expression of the expression vector, 3) separation of pyruvate optical sensors.

The terms “nucleic acid” or “nucleotide” or “polynucleotide” or “nucleic acid sequence” used herein may be in the form of DNA or in the form of RNA. DNA form includes cDNA, genomic DNA, or artificially synthesized DNA. DNA can be single stranded or double stranded. DNA can be either the coding or noncoding strand. When referring to nucleic acids, the term “variant” used herein may be a naturally occurring allelic variant or a non-naturally occurring variant. These nucleotide variants include degenerate variants, substitution variants, deletion variants, and insertion variants. The nucleic acid of the invention may comprise a nucleotide sequence having sequence identity of at least about 50%, at least about 60%, at least about 70%, at least about 75%, at least about 80%, at least about 85%, at least about 90%, at least about 95%, at least about 98%, at least about 99%, or 100% to the nucleic acid sequence. The invention also relates to a nucleic acid fragment that hybridizes to the sequences described above. In an exemplary embodiment, the nucleic acid sequence is shown in SEQ ID NO: 31, which represents the coding sequence of a sensor with cpYFP inserted into position 192/194 of the functional fragment of pyruvate synthase and having the M194V mutation. As used herein, a “nucleic acid fragment” is at least 15 nucleotides in length, preferably at least 30 nucleotides, more preferably at least 50 nucleotides, and even more preferably at least 100 nucleotides or more. The nucleic acid fragment can be used in amplification techniques of nucleic acids (such as PCR).

The full-length sequences or fragments thereof of the optical sensors or fusion proteins of the invention can often be obtained by PCR amplification, artificial synthesis or recombinant procedures. For PCR amplification, primers can be designed according to the nucleotide sequences disclosed in the invention and the sequences in interest can be amplified with a commercially available cDNA library or a cDNA library prepared by conventional methods known to those skilled in the art as template. When the nucleotide sequence is greater than 2500 bp, it is preferable to perform 2˜6 rounds of PCR amplification, and then the individually amplified fragments are spliced together in the correct order. There is no special limit to the procedures and systems for PCR amplification described herein, and conventional PCR amplification procedures and systems in the art may be used. Recombination can also be used to obtain relevant sequences in bulk. This is usually by cloning into a vector, retransferring into cells, and then isolating and purifying the polypeptide or protein in inserest from proliferated host cells by conventional methods. In addition, synthetic methods are also available to synthesize the the sequence in interest, especially for short fragments. In the present invention, when the nucleotide sequence of the optical sensor is less than 2500 bp, it may be synthesized by an artificial synthetic method. The artificial synthetic method is a conventional artificial synthetic method for DNA in the art with no other special requirements. In general, many small fragments are first synthesized, and then ligated to obtain a very long sequence. The DNA sequences of the polypeptides of the invention can also be obtained entirely by chemical synthesis. This DNA sequence can then be introduced into a variety of existing DNA molecules known in the art, such as vectors, and into cells. Mutations can be introduced into the protein sequence of the invention by methods such as mutation PCR or chemical synthesis.

The invention also provides a detection test kit comprising an optical sensor or fusion polypeptide or polynucleotide described herein, or an optical sensor or fusion polypeptide prepared according to the method described herein. The kit also optionally contains other reagents required for the detection of pyruvate using an optical sensor. Such other reagents are routinely known in the art.

The invention also relates to a nucleic acid construct containing the polynucleotides described herein, and one or more regulatory sequences operably linked to such sequences. The polynucleotides described herein can be manipulated in a variety of ways to guarantee expression of said polypeptide or protein. The nucleic acid construct can be manipulated according to the difference or requirements of the expression vector prior to insertion of the nucleic acid construct into the vector. Techniques that utilize recombinant DNA methods to alter polynucleotide sequences are known in the art.

In certain embodiments, the nucleic acid construct is a vector. The vector can be a cloning vector, an expression vector, or a gene knock-in vector, such as a homologous recombination vector. The polynucleotides of the present invention can be cloned into many types of vectors, e.g., plasmids, phagemids, phage derivatives, animal viruses, and cosmids. Cloning vectors can be used to provide coding sequences for proteins or polypeptides of the invention. Expression vectors can be provided to cells in the form of bacterial or viral vectors. Expression of the present polynucleotides is generally achieved by operably linking the polynucleotides of the invention to a promoter and incorporating the construct into an expression vector. This vector may be suitable for replicating and integrating eukaryotic cells. A typical expression vector contains an expression control sequence that can be used to regulate the expression of the desired nucleic acid sequence. Knock-in vectors are used to knock-in or integrate the expression cassettes described herein into the host genome.

The term “expression control sequence” used herein refers to elements that regulate the transcription, translation, and expression of a gene of interest and can be operably linked to the gene of interest, which may be a replication origin, promoter, marker gene, or translational control element, including enhancers, operons, terminators, ribosome binding sites, etc., and the choice of expression control sequence depends on the host cell used. In recombinant expression vectors, “operable ligation” refers to the attachment of the nucleotide sequence of interest to a regulatory sequence in a manner that allows the expression of the nucleotide sequence. Those skilled in the art are well known of methods for constructing expression vectors containing the present fusion protein coding sequences and suitable transcription/translation control signals. These include in vitro recombinant DNA techniques, DNA synthesis techniques, in vivo recombination techniques, etc. Said DNA sequence can be effectively ligated to an appropriate promoter in an expression vector to direct mRNA synthesis. Representative examples of these promoters are: the lac or trp promoter of E. coli; λ-phage PL promoter; eukaryotic promoters including the CMV immediate early promoter, the HSV thymidine kinase promoter, the early and late SV40 promoters, the LTR of retroviruses, and several other promoters known to control gene expression in prokaryotic or eukaryotic cells or their viruses. The expression vector also includes a ribosome binding site for translation initiation and a transcription terminator. In one embodiment, the expression vector may use a commercially available pRESTb vector with no other special requirements. Exemplarily, BamHI and EcoRI were employed to double digest the nucleotide sequence encoding the optical sensor and the expression vector, respectively, and then the enzymatic cleavage products of both were ligated to give recombinant expression vectors. The present invention makes no special limitation to the specific steps and parameters of enzymatic cleavage and ligation, and those conventional in the art would work.

After a recombinant expression vector is obtained, this vector is transformed into host cells to produce a protein or peptide including the fusion protein. This transfer process can be performed with conventional techniques well known to those skilled in the art, such as transformation or transfection. The host cells described herein refer to cells capable of receiving and accommodating recombinant DNA molecules, which are sites of recombinant gene amplification, and ideally the recipient cell should satisfy both conditions of easy access and proliferation. The “host cells” of the invention may include prokaryotic cells and eukaryotic cells, specifically including bacterial cells, yeast cells, insect cells, and mammalian cells. The host cells are said to prefer a variety of cells favouring the expression or fermentative production of the gene product, such cells being well known and commonly used in the art. Those skilled in the art well know how to select appropriate vectors, promoters, enhancers, and host cells.

The methods of transfer to host cells described herein are conventional methods in the art, including calcium phosphate or calcium chloride coprecipitation, DEAE-mannan-mediated transfection, lipofection, naturally competent cells, chemically mediated transfer, or electroporation. When the host is a prokaryote such as E. coli, the process described is preferably CaCl₂ method or MgCl₂ method, and their steps used are well known in the art. When the host cell is a eukaryotic cell, the following DNA transfection methods are used: calcium phosphate coprecipitation, conventional mechanical methods such as microinjection, electroporation, liposome packaging, etc.

After transferring the expression vector into host cells, the host cells transferred with the expression vector are subjected to expansion and expression culture, and the pyruvate optical sensor is isolated. The host cells are expanded for expression by conventional methods. Depending on the host cell species used, the medium used in culture may be various conventional media. Culture is performed under conditions suitable for host cell growth.

In the present invention, an optical sensor is expressed intracellularly, on the cell membrane, or secreted outside the cell. Recombinant proteins can be isolated or purified by various separation methods, if desired, using their physical, chemical, and other properties. The present invention does not have a special limit to a process for separating said pyruvate fluorescent proteins, and a conventional method for the isolation of fusion proteins in the art would work. These methods are well known to those skilled in the art and include, but are not limited to: conventional renaturation treatments, salting out methods, centrifugation, osmotic disruption, sonication, ultra centrifugation, molecular sieving chromatography, adsorption chromatography, ion exchange chromatography, high performance liquid chromatography (HPLC) and other liquid chromatography techniques and the combination of these methods. In one embodiment, His tag affinity chromatography is utilized for optical sensor isolation.

The invention also provides use of said pyruvate optical sensors in real-time localization, quantitative detection of pyruvate as well as high-throughput compound screening. In one aspect, said pyruvate optical sensor, preferably linked with signal peptides at different parts of the cell, is transferred into cells for real-time localization of pyruvate by detecting the strength of fluorescence signals in cells; corresponding quantitative detection of pyruvate is performed by a pyruvate standard titration curve. The pyruvate standard titration curve described herein is plotted from the fluorescence signals of a pyruvate optical sensor in the presence of different concentrations of pyruvate. The pyruvate optical sensor described herein is directly transferred into cells, which is more accurate without time-consuming sample processing process during the real-time localization and quantitative detection of pyruvate. The present pyruvate optical sensor, when performing high-throughput compound screening, adds different compounds to the cell culture fluid, determines changes in pyruvate content, and thus screens out compounds that influence the changes in pyruvate content. The use of the pyruvate optical sensors described herein in real-time localization, quantitative detection of pyruvate as well as high-throughput compound screening are all non-diagnostic and therapeutic purposes, not related to the diagnosis and treatment of diseases.

Concentrations, contents, percentages, and other values may be expressed with ranges available herein. It is also understood that use of these ranges is only for convenience and conciseness, which should be interpreted elastically to include values explicitly mentioned in the upper and lower limits of the range, but also to include all individual values or sub ranges included in the ranges.

### EXAMPLE

The pyruvate optical sensors provided by the invention are described in detail below in combination with the examples, but they cannot be understood as defining the scope of protection of the invention.

I. Experimental Materials and Reagents

The conventional molecular biology cloning methods for genetic engineering and cell culture and imaging methods mainly used in examples are well known to those skilled in the art, for example, Roskams, J., Molecular Biology Laboratory Handbook; J. Sambrook, D. W. Russell ed, Molecular Cloning: A Laboratory Manual, translated by Peitang Huang et al. (3rd edition, August 2002, Science Press); R. I. Freshney et al., Culture of Animal Cells: a Manual of Basic Technique (5th edition), translated by Jingbo Zhang and Cunquan Xu; Juan S. Bonifacino, M. Daso, et al., Short Protocols in Cell Biology, translated by Jingbo Zhang et al.

The pRSETb-cpYFP based, pRSETb-pyruvate binding protein plasmid used in examples was constructed by the Protein Laboratory of East China University of Science and Technology, and the pRSETb plasmid vector was purchased from Invitrogen. All the primers used for PCR were synthesized, purified, and identified to be correct by mass spectrometry (MS) by Shanghai Generay Biotech Co., Ltd. The expression plasmids constructed in examples were all sequenced by BGI and Jie Li Sequencing. For each example, Taq DNA polymerase was purchased from Dongsheng Bio, pfu DNA polymerase was purchased from Tiangen Biochemical Technology (Beijing) Co., Ltd, primeSTAR DNA polymerase was purchased from Takara, and all three polymerases were purchased with corresponding polymerase buffer and dNTPs. Restriction enzymes such as BamHI, BglII, HindIII, NdeI, XhoI, EcoRI, SpeI, T4 ligase, and T4 phosphorylase (T4 PNK) were purchased from Fermentas with corresponding buffers and the like. Transfection reagent Lip2000 Kit was purchased from Invitrogen Amino acids such as pyruvate were purchased from Sigma. Chemical reagents such as inorganic salts were purchased from Sigma-Aldrich unless specifically stated. HEPES salts, ampicillin (Amp) and puromycin were purchased from Ameresco. The 96-well detection blackboard and the 384-well fluorescence detection blackboard were purchased from Grenier Company.

The DNA purification kit used in examples was purchased from BBI (Canada), and the common Plasmid Mini Preparation Kit was purchased from Tiangen Biochemical Technology (Beijing) Co., Ltd. Clonal strain Machl was purchased from Invitrogen. Nickel column affinity chromatography columns and desalting column packing were from GE Healthcare.

The main instruments used in examples include: Biotek Synergy 2 multi-purpose microplate reader (Bio-Tek, USA), X-15R high-speed refrigerated centrifuge (Beckman, USA), Microfuge22R tabletop high-speed refrigerated centrifuge (Beckman, USA), PCR amplimer (Biometra, Germany), ultrasonic disruptor (Ningbo SCIENTZ), nucleic acid electrophoresis instrument (Shennengbocai company), spectrofluorometer (Varian, USA), CO₂ constant temperature cell culture incubator (SANYO), and inverted fluorescence microscope (Nikon, Japan).

II. Molecular Biology Methods and Cellular Experimental Methods

II.1 Polymerase Chain Reaction (PCR):

1. PCR Amplification of Target Fragments:

This method was mainly used for gene fragment amplification and colony PCR to identify positive clones. The reaction system for PCR amplification was as follows: template sequence 0.5-1 the forward primer (25 μM) 0.5 μl, the reverse primer (25 μM) 0.5 μl, 10×pfu buffer 5 μl, pfu DNA polymerase 0.5 μl, dNTP (10 mM) 1 μl, sterilized ultrapure water (ddH2O) 41.5-42 μl, to a total volume of 50 μl. The PCR amplification program was as follows: denaturation at 95 ° C. for 2-10 min, 30 cycles (94-96 ° C. for 30-45 s, 50-65 ° C. for 30-45 s, 72 ° C. for a period (600 bp/min)), and extension at 72 ° C. for 10 min.

2. PCR Amplification of Long Fragments (>2500 bp):

Long fragment amplification, primarily inverse PCR amplification vector, used in the invention is a technique used to obtain site directed mutations in examples below. Reverse PCR primers were designed at the variation site, where the 5′ end of one primer contained the variant nucleotide sequence. Amplified products contained the corresponding mutation site. The reaction system of amplification PCR of long fragments were as follows: template sequence (10 μg-1 ng) 1 μl, the forward primer (25 μM) 0.5 μl, the reverse primer (25 μM) 0.5 μl, 5×PrimerSTAR buffer 10 μl. PrimerSTAR DNA polymerase 0.5 dNTPs (2.5 mM) 4 μl, sterilized ultrapure water (ddH₂O) 33.5 to a total volume of 50 μl. The PCR amplification program was as follows: denaturation at 95 ° C. for 5 min, 30 20 cycles (98 ° C. for 10 s, 50-68 ° C. for 5-15 s, 72 ° C. for a period (1000 bp/min)), and extension at 72 ° C. for 10 min; or denaturation at 95 ° C. for 5 min, 30 cycles (98 ° C. for 10 s, 68 ° C. for a period (1000 bp/min)), and extension at 72 ° C. for 10 min.

II.2 Endonuclease Digestion Reaction:

The system for double digestion of plasmid vectors was as follows: plasmid vector 20 μl (approximately 1.5 μg), 10><Buffer 5 restriction endonuclease 11-2 μl, restriction endonuclease 21-2 μl, made up to a total volume of 50 μl with sterilized ultrapure water. Reaction condition is 37 ° C. for 1-7 hours.

II.3 5′ phosphorylation reaction of DNA fragments

The ends of plasmids or genomes extracted from microorganisms contain phosphate groups, but PCR products do not contain. Therefore, it is necessary to perform phosphate group addition reaction on the 5′-end bases of PCR products, only DNA molecules containing phosphate groups at the ends can occur ligation reaction. The phosphorylation reaction system is as follows: PCR product fragment DNA sequence 5-8 μL, 10×T4 polynucleotide buffer 1 μL, T4 polynucleotide (T4 μL 1 μL), phosphorylation reaction ligation ultrapure water 0-3 μL, total volume 10 μL. The reaction conditions were 37° C., inactivation at 72° C. for 20 minutes after 30 minutes to 2 hours.

II.4 Ligation of Target Fragments and Vectors

There are differences in the ligation method between different fragments and vectors, and three ligation methods are used in this invention.

1. Ligation of Blunt End Short Fragments and Linearized Vectors

The principle of this method is that the blunt end product obtained by PCR, after phosphorylation of the 5′ end of the DNA fragment by T4 PNK, is ligated with a linearized vector in the presence of PEG4000 and T4 DNA ligase to obtain a recombinant plasmid. The homologous recombination ligation system was as follows: T4 PNK treated DNA fragment 4 μl, linearized vector fragment 4 μl, PEG4000 1 μl, 10×T4 ligase buffer 1 μl, T4 DNA ligase 1 μl, to total of 10 μl. The reaction condition was 22° C. for 30 min.

2. Ligation of DNA Fragments Containing Cohesive Ends and Vector Fragments Containing Cohesive Ends

DNA fragments cut by restriction endonucleases usually produce sticky ends that stick out, so they can be ligated with vector fragments containing complementary sticky ends to form recombinant plasmids. The ligation reaction system is as follows: 1-7 μL of PCR product fragment DNA after digestion, 0.5-7 μL of plasmid after digestion, 1 μL of 10×T4 ligase buffer, 1 μL of T4 DNA ligase, and sterilized ultrapure water to make up the total volume 10 μL. Reaction condition was 16° C., 4-8 hours. The mass ratio of PCR product fragments to vector double-enzyme digestion products is roughly between 2:1 and 6:1.

3. Ligation Reactions in Which the Products of DNA Fragments Phosphorylated at the 5′ End Were Self Circularized After Site Directed Mutagenesis Were Introduced by Inverse PCR

The 5′-end phosphorylated DNA fragment is ligated to the 3′-end and 5′-end of the linearized vector through self-circularization ligation reaction to obtain a recombinant plasmid. The self-cyclization ligation reaction system is as follows: phosphorylation reaction system 10 μL, T4 ligase (5 U/μL) 0.5 μL, total volume 10.5 μL. Reaction condition was 16° C., 4-16 hours.

II.5 Preparation and Transformation of Competent Cells

Preparation of Competent Cells:

1. A single colony (e.g., Mach1) is picked and inoculated in 5 ml LB medium at 37° C. on a shaker overnight. 2. 0.5-1 ml of overnight cultured broth was taken and transferred into 50 ml LB medium and incubated at 37 ° C. at 220 rpm for 3 to 5 hours until the OD600 reached 0.5. 3. The cells were precooled in ice bath for 2 hours. 4. 4 oC, centrifugation at 4000 rpm for 10 min. 5. The supernatant was discarded, the cells were resuspended with 5 ml of precooled buffer, and resuspension buffer was added again after homogenization to a final volume of 50 ml. 6. Ice bath for 45 min. 7. 4° C., centrifugation at 4000 rpm for 10 min, the bacteria were resuspended with 5 ml ice precooled storage buffer. 8. 100 μl of broth was loaded to each EP tube and frozen at −80° C. or in liquid nitrogen.

Among them, resuspension buffer: CaCl₂ (100 mM), MgCl₂ (70 mM), NaAc (40 mM) Storage buffer: 0.5 mL DMSO, 1.9 mL 80% glycol, 1 mL 10×CaCl₂ (1 M), 1 mL 10×MgCl₂ (700 mM), 1 mL 10×NaAc (400 mM), 4.6 mL ddH2O.

Transformation of Compenent Cells:

1. Take 100 μl competent cells were taken and thawed on an ice bath. 2. The appropriate volume of ligation product was added, mixed by gently pipetting, and incubated in ice bath for 30 min. The volume of ligation product normally added was less than 1/10 of the volume of competent cells. 3. The broth was put into a 42° C. water bath for heat shock for 90 s and quickly transferred to an ice bath for 5 min. 4. 500 μl LB was added and incubated at 200 rpm on a constant temperature shaker at 37° C. for 1 hour. 5. The broth was centrifuged at 4000 rpm for 3 min and 200 μl supernatant was added to mix the bacteria well and spread evenly on the surface of the agar plate containing appropriate antibiotics, and the plate is inverted in a 37° C. incubator overnight.

II.6 Expression, Purification, and Fluorescent Detection of Proteins

1. Transform an expression vector (such as a pRSETb-based pyruvate optical sensor expression vector) into JM109 (DE3) cells, invert overnight culture, pick clones from the plate into a 250 ml Erlenmeyer flask, and place at 37 Shaker at ° C., culture at 220 rpm to OD=0.4-0.8, add 1/1000 (v/v) IPTG (1 M), and induce expression at 18° C. for 24-36 hours.

2. Upon completion of inducible expression, cells were harvested by centrifugation at 4000 rpm for 30 min, resuspended in 50 mM phosphate buffer and disrupted ultrasonically until the broth were clear, centrifuged at 9600 rpm at 4° C. for 20 min. Centrifuge at 9600 rpm, 4° C. for 20 minutes.

3. The centrifuged supernatant was used to obtain protein by purification through a self packed nickel column affinity chromatography column, and the protein obtained after nickel column affinity chromatography was further used to obtain protein dissolved in 20 mM MOPS buffer (pH 7.4) or phosphate buffer PBS.

4. After the purified protein was identified by SDS-PAGE, the sensor was diluted with assay buffer (100 mM HEPES, 100 mM NaCl, pH 7.3) or phosphate buffered saline PBS to a final concentration of 5-10 μM protein solution. Pyruvate was formulated as a final 1 M stock solution in assay buffer (20 mM MOPS, pH 7.4) or phosphate buffered saline PBS.

5. 100 μl of 1 μM of protein solution was taken, incubated at 37° C. for 5 min, pyruvate was added to mix to a final concentration of 100 mM, respectively, the optical absorption of the protein at 340 nm was determined using a multifunctional fluorescence microplate reader.

6. Take 100 μl of 1 μM protein solution, incubate at 37° C. for 5 minutes, add pyruvate for titration, and measure the fluorescence intensity of the protein emitted at 528 nm after fluorescence excitation at 485 nm. The fluorescence excitation and emission measurement of the samples were completed by a multifunctional fluorescence microplate reader.

7. Take 100 μl of 1 μM protein solution, incubate at 37° C. for 5 minutes, add pyruvate, and measure the absorption and fluorescence spectra of the protein. The determination of the absorption spectrum and fluorescence spectrum of the sample is completed by a spectrophotometer and a fluorescence spectrophotometer.

II.6 Expression, Purification, and Fluorescent Detection of Proteins

1. The pCDNA3.1+-based pyruvate optical sensor plasmid was transfected into HeLa by the transfection reagent Lipofectamine2000 (Invitrogen), and cultured in a cell incubator at 37° C., 5% CO₂. Fluorescence detection was performed 24-36 h after the exogenous gene was fully expressed.

2. After induction of expression, adherent HeLa cells were washed three times with PBS and placed in HBSS solution for fluorescence microscopy and microplate reader assays, respectively.

### Example 1

### Pyruvate Binding Protein Plasmid

The PdhR (96-254) gene in the E. coli gene was amplified by PCR, and the PCR product was recovered after gel electrophoresis and digested with BamHI and HindIII. After ligation with T4 DNA ligase, the product was transformed into MachI, and the transformed MachI was spread on an LB plate (streptomycin sulfate 50 ug/mL) and incubated at 37° C. overnight. After plasmid extraction of the growing MachI transformants, PCR identification was performed. Positive plasmids were sequenced to be correct for subsequent plasmid construction.

### Example 2

### Expression and Detection of Optical Sensors with cpYFP Inserted at Different Sites

In this example, based on pCDFDuet-PdhR(96-254), the insertion sites of cpYFP at the following sites was selected according to the crystal structure of pyruvate-binding protein to obtain the corresponding pCDFDuet-PdhR(96-254)-cpYFP plasmid: 117/118, 117/119, 117/120, 117/121, 118/119, 118/120, 118/121, 119/120, 119/121, 120/121, 140/141, 140/142, 140/143, 141/142, 141/143, 142/143, 160/161, 160/162, 160/163, 160/164, 161/162, 161/163, 161/164, 162/163, 162/164, 163/164, 174/175, 174/176, 175/176, 191/192, 191/193, 191/194, 191/195, 192/193, 192/194, 192/195, 193/194, 193/195, 194/195, 210/211, 210/212, 210/213, 210/214, 211/212, 211/213, 211/214, 212/213, 212/214 or 213/214. Exemplary sequences are shown in Table 1.

TABLE 1
Sequences of optical sensors

| Sequences | Insertion site |
|---|---|
| SEQ ID NO: 6 | 117/121 |
| SEQ ID NO: 7 | 141/143 |
| SEQ ID NO: 8 | 191/192 |
| SEQ ID NO: 9 | 191/193 |
| SEQ ID NO: 10 | 191/194 |
| SEQ ID NO: 11 | 191/195 |
| SEQ ID NO: 12 | 192/193 |
| SEQ ID NO: 13 | 192/194 |
| SEQ ID NO: 14 | 192/195 |
| SEQ ID NO: 15 | 193/194 |
| SEQ ID NO: 16 | 193/195 |
| SEQ ID NO: 17 | 194/195 |
| SEQ ID NO: 18 | 210/214 |

The DNA fragment of cpYFP was generated by PCR, and the linearized pCDFDuet-PdhR (96-254) vector containing different break sites was generated by inverse PCR amplification, and the linearized pCDFDuet-PdhR (96-254) and cpYFP fragments were ligated under the action of homologous recombinase to generate recombinant plasmids, and positive clones were selected by colony PCR and sequenced by Shanghai Jie Li Biotechnology Co., Ltd.

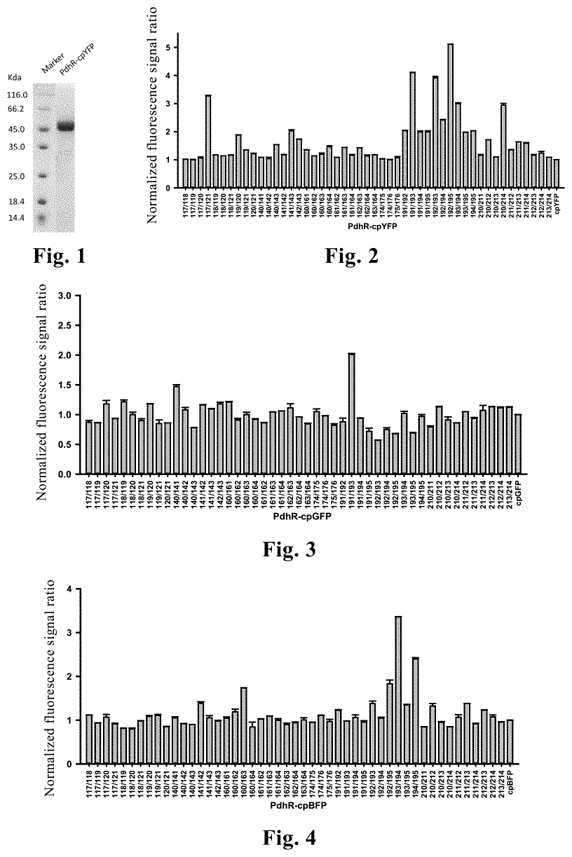

After being sequenced correctly, the recombinant plasmid was transformed into JM109(DE3) to induce expression, and the protein was purified, and the size was around 47.5 Kda by SDS-PAGE. This size corresponds to the size of the PdhR(96-254)-cpYFP fusion protein containing His-tag purified label expressed from pCDFDuet-PdhR(96-254)-cpYFP. The results are shown in FIG. 1.

The purified PdhR(96-254)-cpYFP fusion protein was screened for pyruvate response, and the detection signal of the fusion fluorescent protein containing 100 mM pyruvate was divided by the detection signal of the fusion fluorescent protein without pyruvate. The results are shown in FIG. 2. The detection results show that the optical sensors that respond more than 2-fold to pyruvate than control are those having insertion sites of 117/121, 141/143, 191/192, 191/193, 191/194, 191/195, 192/193, 192/194, 192/195, 193/194, 193/195, 194/195 and 210/214 or with the insertion at corresponding amino acid sites of proteins of the same family

### Example 3

### Expression and Detection of cpGFP Optical Sensors with Different Insertion Sites

According to the method in Example 2, cpYFP was replaced with cpGFP to construct a pyruvate green fluorescent protein fluorescent sensor. As shown in FIG. 3, the detection results showed that the optical sensors that responded more than 2 times to pyruvate included those having insertion sites of 191/193 or with the insertion at corresponding amino acid sites of proteins of the same family

### Example 4

### Expression and Detection of Optical Sensors Having cpBFP Inserted at Different Sites

According to the method in Example 2, cpYFP was replaced with cpBFP to construct a pyruvate blue fluorescent protein fluorescent sensor. As shown in FIG. 4, the detection results showed that the optical sensors that responded more than 2 times to pyruvate included those having insertion sites of 193/194 and 194/195 or with the insertion at corresponding amino acid sites of proteins of the same family

### Example 5

### Expression and Detection of Optical Sensors Having cpmApple Inserted at Different Sites

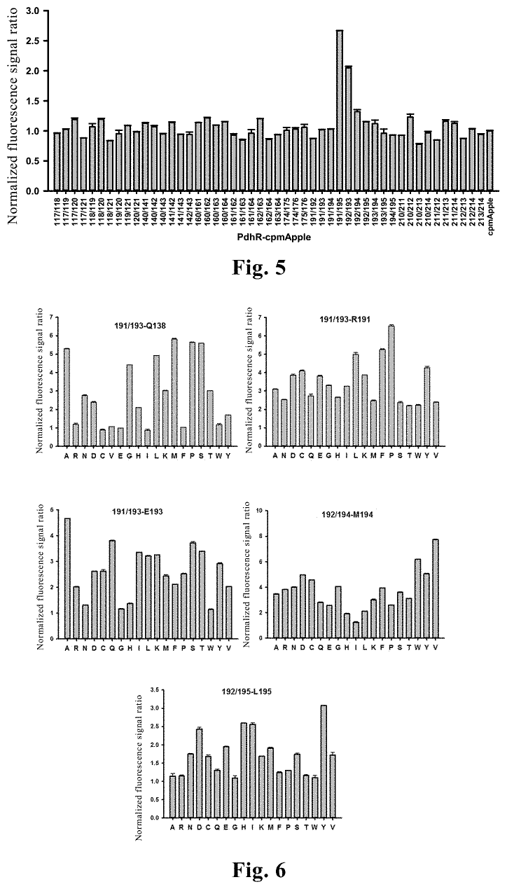

According to the method in Example 2, cpYFP was replaced with cpmApple to construct a pyruvate red fluorescent protein fluorescent sensor. As shown in FIG. 5, the detection results showed that the optical sensors that responded more than 2 times to pyruvate included those having insertion sites of 191/195 and 192/193 or with the insertion at corresponding amino acid positions of proteins of the same family

The results of Examples 2-5 shows that simply replacing the fluorescent proteins cpYFP, cpGFP, cpBFP or cpmApple to obtain pyruvate-responsive sensors of different colors is not feasible.

### Example 6

### Expression and Detection of Mutated cpYFP Optical Sensors

Optical sensor mutants were constructed on the basis of PdhR(96-254)-191/193 -cpYFP, PdhR(96-254)-192/194-cpYFP and PdhR(96-254)-192/195-cpYFP. The plasmid pCDFDuet-PdhR(96-254)-191/193-cpYFP was linearized by inverse PCR with primers having the nucleotide sequence of the desired mutation, phosphorus was added to the obtained PCR products under the action of PNK, T4 DNA ligase and PEG4000 to obtain plasmids with saturation point mutation at the three sites of Q138, R191 and E193. Using the same method, we obtained plasmids with saturation point mutation at M194 based on pCDFDuet-PdhR(96-254)-192/194-cpYFP, and a plasmids with saturation point mutation at L195 based on pCDFDuet-PdhR(96-254)-192/195-cpYFP. On the basis of PdhR(96-254)-192/194-M194V-cpYFP and PdhR(96-254)-141/143-cpYFP, random mutation libraries of sites 190, 191 and 192 were constructed respectively. Sequencing was completed by Shanghai Jie Li Biotechnology Co., Ltd. The sequences of some mutated optical sensors are shown in Table 2. An exemplary nucleic acid sequence is set forth in SEQ ID NO:31 (192/194-M194V).

TABLE 2
Sequences of mutated optical sensors

| | Insertion | |
|---|---|---|
| Sequences | site | Mutation |
| SEQ ID NO: 19 | 191/193 | Q138 mutated to A, N, D, G, H, L, K, |
| | | M, P, S or T |
| SEQ ID NO: 20 | 191/193 | R191 mutated to A, N, D, C, Q, E, G, |
| | | H, I, L, K, M, F, P, S, T, W, Y or V |
| SEQ ID NO: 21 | 191/193 | E193 mutated to A, R, D, C, Q, I, L, |
| | | K, M, F, P, S, T, Y or V |
| SEQ ID NO: 22 | 192/194 | M194 mutated to A, R, N, D, C, Q, E, |
| | | G, L, K, F, P, S, T, W, Y or V) |
| SEQ ID NO: 23 | 192/195 | L195 mutated to D, H, I or Y |
| SEQ ID NO: 24 | 192/194 | M194V/S190E/R191N/R192D |
| SEQ ID NO: 25 | 192/194 | M194V/S190D/R191Y/R192T |
| SEQ ID NO: 26 | 141/143 | S190P/R191H/R192P |
| SEQ ID NO: 27 | 141/143 | S190R/R191S/R192P |
| SEQ ID NO: 28 | 141/143 | S190L/R191V |
| SEQ ID NO: 29 | 141/143 | S190T/R191Q/R192E |
| SEQ ID NO: 30 | 141/143 | R191S/R192T |

The results are shown in FIG. 6. The results of fluorescence detection showed that multiple mutants after saturation mutation at five sites, Q138, R191, E193, M194 and L195, had an enhanced response to pyruvate, which indicates that these five sites are very important for pyruvate binding. In addition, random mutation results of three sites S190, R191 and R192 show that some mutants of these three sites enhance the response to pyruvate, therefore these three sites are also important for pyruvate binding.

### Example 7

### Titration Curve of Pyruvate Optical Sensor

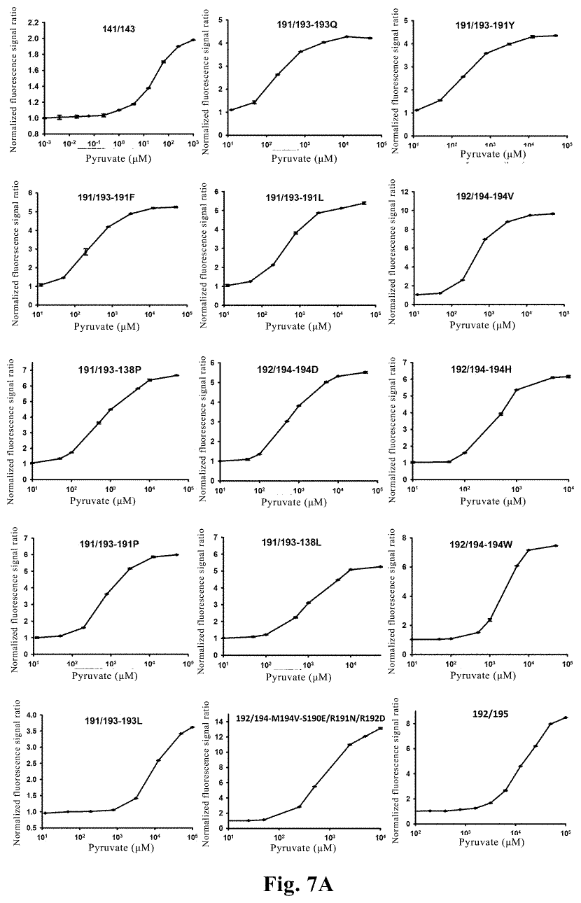

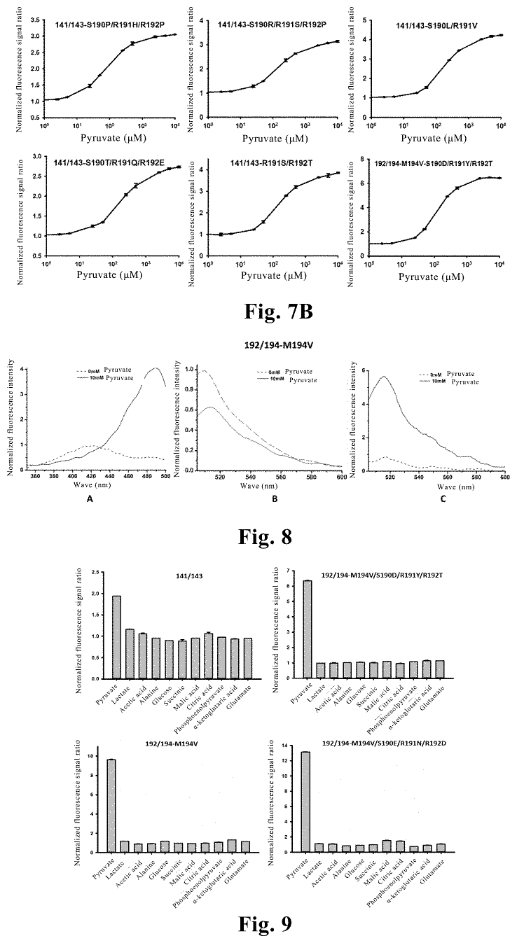

Part of the pyruvate optical sensors obtained in Example 2 and Example 6, i.e. 141/143, 191/193-E193Q, 191/193-R191Y, 191/193-R191F, 191/193-R191L, 192/194-M194V, 191/193-Q138P, 192/194-M194D, 192/194 -M194H, 191/193-R191P, 191/193-Q138L, 192/194-M194W, 191/193-E193L, 192/194-M194V/S190E/R191N/R192D, 192/195, 141/143-S190P/R191H/R192P, 141/143-S190R/R191S/R192P, 141/143-S 190L/R191V, 141/143-S190T/R191Q/R192E, 141/143-R191S/R192T, 141/143-S190D/R191Y/R192T, were subjected to concentration gradient pyruvate detection, and the change of the ratio of fluorescence intensity at 528 nm after excitation at 420 nm and fluorescence intensity at 528 nm after excitation at 485 nm was detected. The Kd (binding constant) of 141/143, 191/193-E193Q, 191/193-R191Y, 191/193-R191F, 191/193-R191L, 192/194-M194V, 20 191/193-Q138P, 192/194-M194D, 192/194-M194H, 191/193-R191P, 191/193-Q138L, 192/194-M194W, 191/193-E193L, 192/194-M194V-S190E/R191N/R192D, 192/195 are 28 μM, 195 μM, 222 μM, 422 μM, 463 μM, 475 μM, 599 μM, 664 μM, 728 μM, 799 μM, 1253 μM, 5142 μM, 9255 μM, 1066 μM, 19944 μM, 84 μM, 158 μM , 185 μM, 186 μM, 164 μM and 134 μM, respectively, and the changes are 2.0 times, 4.1 times, 4.3 times, 5.3 times, 5.0 times, 9.7 times, 6.4 times, 5.3 times, 6.1 times, 6.5 times, 5.1 times, 7.2 times, 3.4 times, 13.1 times, 8.3 times, 3.2 times, 3.1 times, 4.2 times, 2.7 times, 3.9 times and 6.4 times, respectively, as shown in FIGS. 7A-7B.

### Example 8

### Spectral Performance and Specificity of Pyruvate Optical Sensors

Exemplarily, after the purified pyruvate optical sensor PdhR(96-254)-192/194-M194V-cpYFP was treated with 0 mM and 10 mM pyruvate for 10 minutes, respectively, the fluorescence spectrum was detected using a fluorescence spectrophotometer.

Determination of excitation spectra: Excitation spectra were recorded with an excitation range of 350 nm to 500 nm and an emission wavelength of 530 nm, with readings every 5 nm. The results show that the sensor has two excitation peaks at about 420 and 490 nm, as shown in FIG. 8A.

Determination of emission spectra: excitation spectra were recorded with an fixed excitation wavelength of 420 nm and 490 nm and an emission range of 505-600 nm, read every 5 nm. The results showed that after the addition of 10 mM pyruvate, the fluorescence intensity of the sensor excited at 420 nm was reduced by 0.6 times, which was 0.6 times that of the addition of 0 mM pyruvate; and the fluorescence intensity of the sensor excited at 490 nm was enhanced by 5.8 times that of the addition of 0 mM pyruvate. As shown in FIGS. 8B and 8C.

The specificity of the purified pyruvate optical sensor PdhR(96-254)-141/143-cpYFP, PdhR(96-254)-192/194-M194V/S190D/R191Y/R192T-cpYFP, PdhR(96-254)-192/194-M194V-cpYFP and PdhR(96-254)-192/194-M194V/S190E/R191N/R192D-cpYFP was determined, and the results show that the sensor has good specificity, as shown in FIG. 9.

### Example 9

### Subcellular Organelle Localization of Optical Sensors

In this example, different localization signal peptides were used to fuse the optical sensors to localize the optical sensors at different organelles.

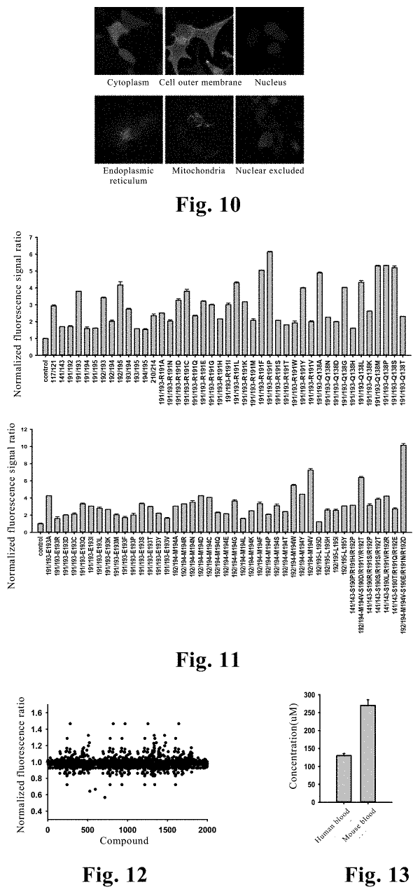

HeLa cells were transfected for 36 hours with optical sensor plasmids fused with different localization signal peptides for 36 hours, rinsed with PBS, placed in HBSS solution, and subjected to fluorescence detection under the FITC channel under an inverted fluorescence microscope. The results are shown in FIG. 10. Pyruvate optical sensors can be localized to subcellular organelles including cytoplasm, outer membrane, nucleus, endoplasmic reticulum, mitochondria, nuclear exclusion by fusing with different specific localization signal peptides. Fluorescence was present in different subcellular structures, and the distribution and intensity of fluorescence varied.

### Example 10

### Intracellular Performance Study of Pyruvate Sensor

Select the samples with ratio485/420 greater than 2 times, transfect HeLa cells for 36 hours with the cytoplasmic-expressed optical sensor plasmid, rinse with PBS, and place the cells in HBSS solution. Then, 10 mM pyruvate was added, and the change in the ratio of the fluorescence intensityat 528 nm after excitation at 420 nm to that at 528 nm after excitation at 485 nm was detected after 30 min. The results are shown in FIG. 11, exogenous addition of pyruvate can cause a rapid response of the sensor in the cytoplasm of HeLa cells.

### Example 11

### Optical Sensor-Based High-Throughput Compound Screening in Living Cells

In this example, we used HeLa cells expressing the pyruvate sensor PdhR(96-254)-192/194-M194V-cpYFP in the cytoplasm for high-throughput compound screening.

Transfected HeLa cells were rinsed with PBS, treated in HBSS solution (without pyruvate) for 1 hr, and then treated with 10 μM of compound for 1 hr. Pyruvate was added dropwise to each sample. Changes in the ratio of fluorescence intensity at 528 nm emission excited at 420 nm to that of 528 nm emission excited at 485 nm were recorded using a microplate reader. Samples not treated with any compound were used as controls for normalization. The results are shown in FIG. 12. Of the 2000 compounds used, the vast majority had minimal effect on the entry of pyruvate into cells. Five compounds were able to increase the cellular uptake of pyruvate, and other 8 compounds were able to decrease the cellular uptake of pyruvate significantly.

### Example 12

### Quantitative Detection of Pyruvate in Blood with Optical Sensors

In this example, we analyzed mouse and human blood supernatants for pyruvate using the purified pyruvate sensor PdhR(96-254)-141/143-cpYFP.

After 10 min of treatment by mixing the pyruvate sensor PdhR(96-254)-141/143-cpYFP with the diluted blood supernatant, the ratio of the fluorescence intensity at 528 nm emission excited at 420nm to that at 528 nm emission excited at 485 nm was detected by a microplate reader. The results are shown in FIG. 13. The pyruvate content were around 270 μM in mouse blood and around 130 μM in human blood.

It can be seen from the above examples that the pyruvate fluorescent sensor provided by the present invention has relatively small protein molecular weight and was prone to maturation, had large dynamic change in fluorescence, good specificity, and ability to be expressed in cells by gene manipulation methods, which could locate, quantitatively detect pyruvate in real time inside and outside cells; and enables high-throughput compound screening.

### OTHER EMBODIMENTS

This specification describes a number of embodiments. It should be understood, however, that various modifications that those skilled in the art will come to know from reading this specification without departing from the spirit and scope of the invention should also be included within the scope of the appended claims.

1. An optical sensor comprising a pyruvate-sensitive polypeptide and an optically active polypeptide, wherein the optically active polypeptide is located in the sequence of the pyruvate-sensitive polypeptide.

2. The optical sensor of claim 1, wherein,
the pyruvate-sensitive polypeptide has the sequence shown in SEQ ID NO: 1 or a functional fragment thereof, or a sequence having 35%, 40%, 50%, 60%, 70%, 80%, 85%, 90%, 95%, 99% sequence identity with it, or
the pyruvate-sensitive polypeptide has amino acids 96-254 of the sequence shown in SEQ ID NO: 1, or a sequence having 35%, 40%, 50%, 60%, 70%, 80%, 85%, 90%, 95%, 99% sequence identity with it.

3. The optical sensor of claim 2, wherein the optically active polypeptide is located in the pyruvate-sensitive polypeptide at a position selected from the group consisting of residues 117-121, 140-143, 160-164, 174-176, 191-195 and/or 210-214.

4-10. (canceled)

11. The optical sensor of claim 3, wherein the optically active polypeptide is located in the pyruvate-sensitive polypeptide at a position selected from the group consisting of residues 117-121, 140-143, 191-195 and 210-214.

12. The optical sensor of claim 3, wherein the optically active polypeptide is located at one or more sites of the pyruvate-sensitive polypeptide selected from the group consisting of: 117/118, 117/119, 117/120, 117/121, 118/119, 118/120, 118/121, 119/120, 119/121, 120/121, 140/141, 140/142, 140/143, 141/142, 141/143, 142/143, 160/161, 160/162, 160/163, 160/164, 161/162, 161/163, 161/164, 162/163, 162/164, 163/164, 174/175, 174/176, 175/176, 191/192, 191/193, 191/194, 191/195, 192/193, 192/194, 192/195, 193/194, 193/195, 194/195, 210/211, 210/212, 210/213, 210/214, 211/212, 211/213, 211/214, 212/213, 212/214 and/or 213/214.

13. The optical sensor of claim 3, wherein, the optically active polypeptide is located in the pyruvate-sensitive polypeptide at one or more sites selected from the group consisting of: 117/121, 141/143, 191/192, 191/193, 191/194, 191/195, 192/193, 192/194, 192/195, 193/194, 193/195, 194/195 or 210/214.

14. The optical sensor of claim 2, wherein, the pyruvate-sensitive polypeptide comprises a mutation at one or more positions selected from the group consisting of: Q138, S190, R191, R192, E193, M194, L195.

15. The optical sensor of claim 14, wherein, the mutation is selected from the group consisting of: Q138S, Q138Y, Q138C, Q138L, Q138P, Q138H, Q138R, Q138W, Q1381, Q138T, Q138N, Q138K, Q138F, Q138V, Q138A, Q138D, Q138E, Q138M, Q138A, S190E, R1915, R191Y, R191C, R191L, R191P, R191H, R191Q, R191W, R1911, R191T, R191N, R191K, R191F, R191V, R191A, R191D, R191E, R191M, R191A, R192D, E193S, E193Y, E193C, E193L, E193P, E193H, E193Q, E193W, E193R, E193I, E193T, E193N, E193K, E193M, E193V, E193F, E193D, E193A, E193G, M194S, M194Y, M194C, M194L, M194P, M194H, M194Q, M194W, M194R, M194I, M194A, M194N, M194K, M194T, M194V, M194F, M194D, M194E, T102G, L195S, L195Y, L195C, L195D, L195P, L195H, L195Q, L195W, L195I, L195T, L195N, L195K, L195R, L195V, L195A, L195F, L195E, L195M and L195A.

16. The optical sensor of claim 15, wherein, the mutation comprises: Q138P, Q138L, R191Y, R191F, R191L, R191P, E193Q, E193L, M194D, M194V, M194H, M194W, M194V/S190E/R191N/R192D, M194V/S190D/R191Y/R192T, S190P/R191H/R192P, S190R/R191S/R192P, S190L/R191V, S190T/R191Q/R192E or R191S/R192T.

17. A nucleic acid sequence selected from the group consisting of
(1) a polynucleotide encoding the optical sensor according to claim 1;
(2) a fragment of (1);
(3) a complement sequence of (1) or (2).

18. A nucleic acid construct comprising the nucleic acid sequence according to claim 17.

19. The nucleic acid construct of claim 18, wherein, the nucleic acid construct is an expression vector.

20. A host cell, wherein the host cell
(1) expresses the optical sensor according to claim 1;
(2) comprises a nucleic acid sequence encoding the optical sensor of (1); or
(3) comprises a nucleic acid construct comprising the nucleic acid sequence of (2).

21. A method for producing the optical sensor according to claim 1, comprising culturing a host cells, and separating said optical sensor from the culture, wherein the host cell
(1) expresses the optical sensor;
(2) comprises a nucleic acid sequence encoding the optical sensor of (1); or
(3) comprises a nucleic acid construct comprising the nucleic acid sequence of (2).

22. A method for detecting pyruvate in a sample, comprising contacting the sample with an optical sensor according to claim 1, and detecting changes in an optically active polypeptide.

23. The method according to claim 22, wherein, the sample is blood.

24. The method according to claim 22, wherein, the detecting is performed in vivo, in vitro, subcellularly, or in situ.

25. A method for screening compounds, comprising contacting a candidate compound with an optical sensor according to claim 1, detecting changes in the optically active polypeptide, and screening the compounds according to the changes in the optically active polypeptide.

26. The method according to claim 25, wherein, the candidate compound is a pharmaceutical.

27. A detection kit comprising:
(1) the optical sensor according to claim 1;
(2) a nucleic acid sequence encoding the optical sensor of (1);
(3) a nucleic acid construct comprising the nucleic acid sequence of (2); or
(4) a cell expressing the optical sensor of (1); and additional reagents required for pyruvate detection by the optical sensors.


SEQUENCE LISTING (31 sequences)

SEQ ID NO: 1 - protein, 254 aa, Escherichia coli [sequence omitted]

SEQ ID NO: 2 - protein, 246 aa, Artificial Sequence
  cpYFP
YNSDNVYIMADKQKNGIKANFKIRHNVEDGSVQLADHYQQNTPIGDGPVLLPDNHYLSFQSVLSKDPNEKRDHMVLLEFVTAAGITLGMDELYNVDGGSGGTGSKGEELFTGVVPILVELDGDVNGHKFSVSGEGEGDATYGKLTLKLICTTGKLPVPWPTLVTTLGYGLKCFARYPDHMKQHDFFKSAMPEGYVQERTIFFKDDGNYKTRAEVKFEGDTLVNRIELKGIDFKEDGNILGHKLEYN

SEQ ID NO: 3 - protein, 241 aa, Artificial Sequence
  cpGFP
NVYIKADKQKNGIKANFKIRHNIEDGGVQLAYHYQQNTPIGDGPVLLPDNHYLSVQSILSKDPNEKRDHMVLLEFVTAAGITLGMDELYKGGTGGSMVSKGEELFTGVVPIQVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYN

SEQ ID NO: 4 - protein, 243 aa, Artificial Sequence
  cpBFP
NVYIKADKQKNGIKANFKIRHNIEGGGVQLAYHYQQNTPIGDGPVLLPDNHYLSVQSILSKDPNEKRDHMVLLEFVTAAGITLGMDELYKGGTGGSESMVSKGEELFTGVVPIQVELDGDVNGHKFSVSGEGEGDATYGKLTLKFICTTGKLPVPWPTLVTTLSHGVQCFSRYPDHMKQHDFFKSAMPGGYIQERTIFFKDDGNYKTRAEVKFEGDTLVNRIELKGIDFKEDGNILGHKLEYN

SEQ ID NO: 5 - protein, 242 aa, Artificial Sequence
  cpmApple
VSERMYPEDGALKSEIKKGLRLKDGGHYAAEVKTTYKAKKPVQLPGAYIVDIKLDIVSHNEDYTIVEQCERAEGRHSTGGMDELYKGGTGGSLVSKGEEDNMAIIKEFMRFKVHMEGSVNGHEFEIEGEGEGRPYEAFQTAKLKVTKGGPLPFAWDILSPQFMYGSKAYIKHPADIPDYFKLSFPEGFRWERVMNFEDGGIIHVNQDSSLQDGVFIYKVKLRGTNFPPDGPVMQKKTMGWEA

SEQ ID NO: 6 - protein, 403 aa, Artificial Sequence
  sensor
MSQYDLLETRHALEGIAAYYAALYNSDNVYIMADKQKNGIKANFKIRHNVEDGSVQLADHYQQNTPIGDGPVLLPDNHYLSFQSVLSKDPNEKRDHMVLLEFVTAAGITLGMDELYNVDGGSGGTGSKGEELFTGVVPILVELDGDVNGHKFSVSGEGEGDATYGKLTLKLICTTGKLPVPWPTLVTTLGYGLKCFARYPDHMKQHDFFKSAMPEGYVQERTIFFKDDGNYKTRAEVKFEGDTLVNRIELKGIDFKEDGNILGHKLEYNDEDKERIRELHHAIELAQQSGDLDAESNAVLQYQIAVTEAAHNVVLLHLLRCMEPMLAQNVRQNFELLYSRREMLPLVSSHRTRIFEAIMAGKPEEAREASHRHLAFIEEILLDRSREESRRERSLRRLEQRKN

SEQ ID NO: 7 - protein, 405 aa, Artificial Sequence
  sensor
MSQYDLLETRHALEGIAAYYAALRSTDEDKERIRELHHAIELAQQSGYNSDNVYIMADKQKNGIKANFKIRHNVEDGSVQLADHYQQNTPIGDGPVLLPDNHYLSFQSVLSKDPNEKRDHMVLLEFVTAAGITLGMDELYNVDGGSGGTGSKGEELFTGVVPILVELDGDVNGHKFSVSGEGEGDATYGKLTLKLICTTGKLPVPWPTLVTTLGYGLKCFARYPDHMKQHDFFKSAMPEGYVQERTIFFKDDGNYKTRAEVKFEGDTLVNRIELKGIDFKEDGNILGHKLEYNLDAESNAVLQYQIAVTEAAHNVVLLHLLRCMEPMLAQNVRQNFELLYSRREMLPLVSSHRTRIFEAIMAGKPEEAREASHRHLAFIEEILLDRSREESRRERSLRRLEQRKN

SEQ ID NO: 8 - protein, 406 aa, Artificial Sequence
  sensor
MSQYDLLETRHALEGIAAYYAALRSTDEDKERIRELHHAIELAQQSGDLDAESNAVLQYQIAVTEAAHNVVLLHLLRCMEPMLAQNVRQNFELLYSRYNSDNVYIMADKQKNGIKANFKIRHNVEDGSVQLADHYQQNTPIGDGPVLLPDNHYLSFQSVLSKDPNEKRDHMVLLEFVTAAGITLGMDELYNVDGGSGGTGSKGEELFTGVVPILVELDGDVNGHKFSVSGEGEGDATYGKLTLKLICTTGKLPVPWPTLVTTLGYGLKCFARYPDHMKQHDFFKSAMPEGYVQERTIFFKDDGNYKTRAEVKFEGDTLVNRIELKGIDFKEDGNILGHKLEYNREMLPLVSSHRTRIFEAIMAGKPEEAREASHRHLAFIEEILLDRSREESRRERSLRRLEQRKN

SEQ ID NO: 9 - protein, 405 aa, Artificial Sequence
  sensor
MSQYDLLETRHALEGIAAYYAALRSTDEDKERIRELHHAIELAQQSGDLDAESNAVLQYQIAVTEAAHNVVLLHLLRCMEPMLAQNVRQNFELLYSRYNSDNVYIMADKQKNGIKANFKIRHNVEDGSVQLADHYQQNTPIGDGPVLLPDNHYLSFQSVLSKDPNEKRDHMVLLEFVTAAGITLGMDELYNVDGGSGGTGSKGEELFTGVVPILVELDGDVNGHKFSVSGEGEGDATYGKLTLKLICTTGKLPVPWPTLVTTLGYGLKCFARYPDHMKQHDFFKSAMPEGYVQERTIFFKDDGNYKTRAEVKFEGDTLVNRIELKGIDFKEDGNILGHKLEYNEMLPLVSSHRTRIFEAIMAGKPEEAREASHRHLAFIEEILLDRSREESRRERSLRRLEQRKN

SEQ ID NO: 10 - protein, 404 aa, Artificial Sequence
  sensor
MSQYDLLETRHALEGIAAYYAALRSTDEDKERIRELHHAIELAQQSGDLDAESNAVLQYQIAVTEAAHNVVLLHLLRCMEPMLAQNVRQNFELLYSRYNSDNVYIMADKQKNGIKANFKIRHNVEDGSVQLADHYQQNTPIGDGPVLLPDNHYLSFQSVLSKDPNEKRDHMVLLEFVTAAGITLGMDELYNVDGGSGGTGSKGEELFTGVVPILVELDGDVNGHKFSVSGEGEGDATYGKLTLKLICTTGKLPVPWPTLVTTLGYGLKCFARYPDHMKQHDFFKSAMPEGYVQERTIFFKDDGNYKTRAEVKFEGDTLVNRIELKGIDFKEDGNILGHKLEYNMLPLVSSHRTRIFEAIMAGKPEEAREASHRHLAFIEEILLDRSREESRRERSLRRLEQRKN

SEQ ID NO: 11 - protein, 403 aa, Artificial Sequence
  sensor
MSQYDLLETRHALEGIAAYYAALRSTDEDKERIRELHHAIELAQQSGDLDAESNAVLQYQIAVTEAAHNVVLLHLLRCMEPMLAQNVRQNFELLYSRYNSDNVYIMADKQKNGIKANFKIRHNVEDGSVQLADHYQQNTPIGDGPVLLPDNHYLSFQSVLSKDPNEKRDHMVLLEFVTAAGITLGMDELYNVDGGSGGTGSKGEELFTGVVPILVELDGDVNGHKFSVSGEGEGDATYGKLTLKLICTTGKLPVPWPTLVTTLGYGLKCFARYPDHMKQHDFFKSAMPEGYVQERTIFFKDDGNYKTRAEVKFEGDTLVNRIELKGIDFKEDGNILGHKLEYNLPLVSSHRTRIFEAIMAGKPEEAREASHRHLAFIEEILLDRSREESRRERSLRRLEQRKN

SEQ ID NO: 12 - protein, 406 aa, Artificial Sequence
  sensor
MSQYDLLETRHALEGIAAYYAALRSTDEDKERIRELHHAIELAQQSGDLDAESNAVLQYQIAVTEAAHNVVLLHLLRCMEPMLAQNVRQNFELLYSRRYNSDNVYIMADKQKNGIKANFKIRHNVEDGSVQLADHYQQNTPIGDGPVLLPDNHYLSFQSVLSKDPNEKRDHMVLLEFVTAAGITLGMDELYNVDGGSGGTGSKGEELFTGVVPILVELDGDVNGHKFSVSGEGEGDATYGKLTLKLICTTGKLPVPWPTLVTTLGYGLKCFARYPDHMKQHDFFKSAMPEGYVQERTIFFKDDGNYKTRAEVKFEGDTLVNRIELKGIDFKEDGNILGHKLEYNEMLPLVSSHRTRIFEAIMAGKPEEAREASHRHLAFIEEILLDRSREESRRERSLRRLEQRKN

SEQ ID NO: 13 - protein, 405 aa, Artificial Sequence
  sensor
MSQYDLLETRHALEGIAAYYAALRSTDEDKERIRELHHAIELAQQSGDLDAESNAVLQYQIAVTEAAHNVVLLHLLRCMEPMLAQNVRQNFELLYSRRYNSDNVYIMADKQKNGIKANFKIRHNVEDGSVQLADHYQQNTPIGDGPVLLPDNHYLSFQSVLSKDPNEKRDHMVLLEFVTAAGITLGMDELYNVDGGSGGTGSKGEELFTGVVPILVELDGDVNGHKFSVSGEGEGDATYGKLTLKLICTTGKLPVPWPTLVTTLGYGLKCFARYPDHMKQHDFFKSAMPEGYVQERTIFFKDDGNYKTRAEVKFEGDTLVNRIELKGIDFKEDGNILGHKLEYNMLPLVSSHRTRIFEAIMAGKPEEAREASHRHLAFIEEILLDRSREESRRERSLRRLEQRKN

SEQ ID NO: 14 - protein, 404 aa, Artificial Sequence
  sensor
MSQYDLLETRHALEGIAAYYAALRSTDEDKERIRELHHAIELAQQSGDLDAESNAVLQYQIAVTEAAHNVVLLHLLRCMEPMLAQNVRQNFELLYSRRYNSDNVYIMADKQKNGIKANFKIRHNVEDGSVQLADHYQQNTPIGDGPVLLPDNHYLSFQSVLSKDPNEKRDHMVLLEFVTAAGITLGMDELYNVDGGSGGTGSKGEELFTGVVPILVELDGDVNGHKFSVSGEGEGDATYGKLTLKLICTTGKLPVPWPTLVTTLGYGLKCFARYPDHMKQHDFFKSAMPEGYVQERTIFFKDDGNYKTRAEVKFEGDTLVNRIELKGIDFKEDGNILGHKLEYNLPLVSSHRTRIFEAIMAGKPEEAREASHRHLAFIEEILLDRSREESRRERSLRRLEQRKN

SEQ ID NO: 15 - protein, 406 aa, Artificial Sequence
  sensor
MSQYDLLETRHALEGIAAYYAALRSTDEDKERIRELHHAIELAQQSGDLDAESNAVLQYQIAVTEAAHNVVLLHLLRCMEPMLAQNVRQNFELLYSRREYNSDNVYIMADKQKNGIKANFKIRHNVEDGSVQLADHYQQNTPIGDGPVLLPDNHYLSFQSVLSKDPNEKRDHMVLLEFVTAAGITLGMDELYNVDGGSGGTGSKGEELFTGVVPILVELDGDVNGHKFSVSGEGEGDATYGKLTLKLICTTGKLPVPWPTLVTTLGYGLKCFARYPDHMKQHDFFKSAMPEGYVQERTIFFKDDGNYKTRAEVKFEGDTLVNRIELKGIDFKEDGNILGHKLEYNMLPLVSSHRTRIFEAIMAGKPEEAREASHRHLAFIEEILLDRSREESRRERSLRRLEQRKN

SEQ ID NO: 16 - protein, 405 aa, Artificial Sequence
  sensor
MSQYDLLETRHALEGIAAYYAALRSTDEDKERIRELHHAIELAQQSGDLDAESNAVLQYQIAVTEAAHNVVLLHLLRCMEPMLAQNVRQNFELLYSRREYNSDNVYIMADKQKNGIKANFKIRHNVEDGSVQLADHYQQNTPIGDGPVLLPDNHYLSFQSVLSKDPNEKRDHMVLLEFVTAAGITLGMDELYNVDGGSGGTGSKGEELFTGVVPILVELDGDVNGHKFSVSGEGEGDATYGKLTLKLICTTGKLPVPWPTLVTTLGYGLKCFARYPDHMKQHDFFKSAMPEGYVQERTIFFKDDGNYKTRAEVKFEGDTLVNRIELKGIDFKEDGNILGHKLEYNLPLVSSHRTRIFEAIMAGKPEEAREASHRHLAFIEEILLDRSREESRRERSLRRLEQRKN

SEQ ID NO: 17 - protein, 406 aa, Artificial Sequence
  sensor
MSQYDLLETRHALEGIAAYYAALRSTDEDKERIRELHHAIELAQQSGDLDAESNAVLQYQIAVTEAAHNVVLLHLLRCMEPMLAQNVRQNFELLYSRREMYNSDNVYIMADKQKNGIKANFKIRHNVEDGSVQLADHYQQNTPIGDGPVLLPDNHYLSFQSVLSKDPNEKRDHMVLLEFVTAAGITLGMDELYNVDGGSGGTGSKGEELFTGVVPILVELDGDVNGHKFSVSGEGEGDATYGKLTLKLICTTGKLPVPWPTLVTTLGYGLKCFARYPDHMKQHDFFKSAMPEGYVQERTIFFKDDGNYKTRAEVKFEGDTLVNRIELKGIDFKEDGNILGHKLEYNLPLVSSHRTRIFEAIMAGKPEEAREASHRHLAFIEEILLDRSREESRRERSLRRLEQRKN

SEQ ID NO: 18 - protein, 403 aa, Artificial Sequence
  sensor
MSQYDLLETRHALEGIAAYYAALRSTDEDKERIRELHHAIELAQQSGDLDAESNAVLQYQIAVTEAAHNVVLLHLLRCMEPMLAQNVRQNFELLYSRREMLPLVSSHRTRIFEAIMYNSDNVYIMADKQKNGIKANFKIRHNVEDGSVQLADHYQQNTPIGDGPVLLPDNHYLSFQSVLSKDPNEKRDHMVLLEFVTAAGITLGMDELYNVDGGSGGTGSKGEELFTGVVPILVELDGDVNGHKFSVSGEGEGDATYGKLTLKLICTTGKLPVPWPTLVTTLGYGLKCFARYPDHMKQHDFFKSAMPEGYVQERTIFFKDDGNYKTRAEVKFEGDTLVNRIELKGIDFKEDGNILGHKLEYNPEEAREASHRHLAFIEEILLDRSREESRRERSLRRLEQRKN

SEQ ID NO: 19 - protein, 405 aa, Artificial Sequence
  sensor
  misc_feature (44)..(44): The ′Xaa′ at location 44 stands for A, N, D, G, H, L, K, M, P, S, or T.
MSQYDLLETRHALEGIAAYYAALRSTDEDKERIRELHHAIELAXQSGDLDAESNAVLQYQIAVTEAAHNVVLLHLLRCMEPMLAQNVRQNFELLYSRYNSDNVYIMADKQKNGIKANFKIRHNVEDGSVQLADHYQQNTPIGDGPVLLPDNHYLSFQSVLSKDPNEKRDHMVLLEFVTAAGITLGMDELYNVDGGSGGTGSKGEELFTGVVPILVELDGDVNGHKFSVSGEGEGDATYGKLTLKLICTTGKLPVPWPTLVTTLGYGLKCFARYPDHMKQHDFFKSAMPEGYVQERTIFFKDDGNYKTRAEVKFEGDTLVNRIELKGIDFKEDGNILGHKLEYNEMLPLVSSHRTRIFEAIMAGKPEEAREASHRHLAFIEEILLDRSREESRRERSLRRLEQRKN

SEQ ID NO: 20 - protein, 405 aa, Artificial Sequence
  sensor
  misc_feature (97)..(97): The ′Xaa′ at location 97 stands for A, N, D, C, Q, E, G, H, I, L, K, M, F, P, S, T, W, Y, or V.
MSQYDLLETRHALEGIAAYYAALRSTDEDKERIRELHHAIELAQQSGDLDAESNAVLQYQIAVTEAAHNVVLLHLLRCMEPMLAQNVRQNFELLYSXYNSDNVYIMADKQKNGIKANFKIRHNVEDGSVQLADHYQQNTPIGDGPVLLPDNHYLSFQSVLSKDPNEKRDHMVLLEFVTAAGITLGMDELYNVDGGSGGTGSKGEELFTGVVPILVELDGDVNGHKFSVSGEGEGDATYGKLTLKLICTTGKLPVPWPTLVTTLGYGLKCFARYPDHMKQHDFFKSAMPEGYVQERTIFFKDDGNYKTRAEVKFEGDTLVNRIELKGIDFKEDGNILGHKLEYNEMLPLVSSHRTRIFEAIMAGKPEEAREASHRHLAFIEEILLDRSREESRRERSLRRLEQRKN

SEQ ID NO: 21 - protein, 405 aa, Artificial Sequence
  sensor
  misc_feature (344)..(344): The ′Xaa′ at location 344 stands for A, R, D, C, Q, I, L, K, M, F, P, S, T, Y, or V.
MSQYDLLETRHALEGIAAYYAALRSTDEDKERIRELHHAIELAQQSGDLDAESNAVLQYQIAVTEAAHNVVLLHLLRCMEPMLAQNVRQNFELLYSRYNSDNVYIMADKQKNGIKANFKIRHNVEDGSVQLADHYQQNTPIGDGPVLLPDNHYLSFQSVLSKDPNEKRDHMVLLEFVTAAGITLGMDELYNVDGGSGGTGSKGEELFTGVVPILVELDGDVNGHKFSVSGEGEGDATYGKLTLKLICTTGKLPVPWPTLVTTLGYGLKCFARYPDHMKQHDFFKSAMPEGYVQERTIFFKDDGNYKTRAEVKFEGDTLVNRIELKGIDFKEDGNILGHKLEYNXMLPLVSSHRTRIFEAIMAGKPEEAREASHRHLAFIEEILLDRSREESRRERSLRRLEQRKN

SEQ ID NO: 22 - protein, 405 aa, Artificial Sequence
  sensor
  misc_feature (345)..(345): The ′Xaa′ at location 345 stands for A, R, N, D, C, Q, E, G, L, K, F, P, S, T, W, Y, or V.
MSQYDLLETRHALEGIAAYYAALRSTDEDKERIRELHHAIELAQQSGDLDAESNAVLQYQIAVTEAAHNVVLLHLLRCMEPMLAQNVRQNFELLYSRRYNSDNVYIMADKQKNGIKANFKIRHNVEDGSVQLADHYQQNTPIGDGPVLLPDNHYLSFQSVLSKDPNEKRDHMVLLEFVTAAGITLGMDELYNVDGGSGGTGSKGEELFTGVVPILVELDGDVNGHKFSVSGEGEGDATYGKLTLKLICTTGKLPVPWPTLVTTLGYGLKCFARYPDHMKQHDFFKSAMPEGYVQERTIFFKDDGNYKTRAEVKFEGDTLVNRIELKGIDFKEDGNILGHKLEYNXLPLVSSHRTRIFEAIMAGKPEEAREASHRHLAFIEEILLDRSREESRRERSLRRLEQRKN

SEQ ID NO: 23 - protein, 404 aa, Artificial Sequence
  sensor
  misc_feature (345)..(345): The ′Xaa′ at location 345 stands for D, H, I, or Y.
MSQYDLLETRHALEGIAAYYAALRSTDEDKERIRELHHAIELAQQSGDLDAESNAVLQYQIAVTEAAHNVVLLHLLRCMEPMLAQNVRQNFELLYSRRYNSDNVYIMADKQKNGIKANFKIRHNVEDGSVQLADHYQQNTPIGDGPVLLPDNHYLSFQSVLSKDPNEKRDHMVLLEFVTAAGITLGMDELYNVDGGSGGTGSKGEELFTGVVPILVELDGDVNGHKFSVSGEGEGDATYGKLTLKLICTTGKLPVPWPTLVTTLGYGLKCFARYPDHMKQHDFFKSAMPEGYVQERTIFFKDDGNYKTRAEVKFEGDTLVNRIELKGIDFKEDGNILGHKLEYNXPLVSSHRTRIFEAIMAGKPEEAREASHRHLAFIEEILLDRSREESRRERSLRRLEQRKN

SEQ ID NO: 24 - protein, 405 aa, Artificial Sequence
  sensor
MSQYDLLETRHALEGIAAYYAALRSTDEDKERIRELHHAIELAQQSGDLDAESNAVLQYQIAVTEAAHNVVLLHLLRCMEPMLAQNVRQNFELLYENDYNSDNVYIMADKQKNGIKANFKIRHNVEDGSVQLADHYQQNTPIGDGPVLLPDNHYLSFQSVLSKDPNEKRDHMVLLEFVTAAGITLGMDELYNVDGGSGGTGSKGEELFTGVVPILVELDGDVNGHKFSVSGEGEGDATYGKLTLKLICTTGKLPVPWPTLVTTLGYGLKCFARYPDHMKQHDFFKSAMPEGYVQERTIFFKDDGNYKTRAEVKFEGDTLVNRIELKGIDFKEDGNILGHKLEYNVLPLVSSHRTRIFEAIMAGKPEEAREASHRHLAFIEEILLDRSREESRRERSLRRLEQRKN

SEQ ID NO: 25 - protein, 405 aa, Artificial Sequence
  sensor
MSQYDLLETRHALEGIAAYYAALRSTDEDKERIRELHHAIELAQQSGDLDAESNAVLQYQIAVTEAAHNVVLLHLLRCMEPMLAQNVRQNFELLYDYTYNSDNVYIMADKQKNGIKANFKIRHNVEDGSVQLADHYQQNTPIGDGPVLLPDNHYLSFQSVLSKDPNEKRDHMVLLEFVTAAGITLGMDELYNVDGGSGGTGSKGEELFTGVVPILVELDGDVNGHKFSVSGEGEGDATYGKLTLKLICTTGKLPVPWPTLVTTLGYGLKCFARYPDHMKQHDFFKSAMPEGYVQERTIFFKDDGNYKTRAEVKFEGDTLVNRIELKGIDFKEDGNILGHKLEYNVLPLVSSHRTRIFEAIMAGKPEEAREASHRHLAFIEEILLDRSREESRRERSLRRLEQRKN

SEQ ID NO: 26 - protein, 405 aa, Artificial Sequence
  sensor
MSQYDLLETRHALEGIAAYYAALRSTDEDKERIRELHHAIELAQQSGYNSDNVYIMADKQKNGIKANFKIRHNVEDGSVQLADHYQQNTPIGDGPVLLPDNHYLSFQSVLSKDPNEKRDHMVLLEFVTAAGITLGMDELYNVDGGSGGTGSKGEELFTGVVPILVELDGDVNGHKFSVSGEGEGDATYGKLTLKLICTTGKLPVPWPTLVTTLGYGLKCFARYPDHMKQHDFFKSAMPEGYVQERTIFFKDDGNYKTRAEVKFEGDTLVNRIELKGIDFKEDGNILGHKLEYNLDAESNAVLQYQIAVTEAAHNVVLLHLLRCMEPMLAQNVRQNFELLYPHPEMLPLVSSHRTRIFEAIMAGKPEEAREASHRHLAFIEEILLDRSREESRRERSLRRLEQRKN

SEQ ID NO: 27 - protein, 405 aa, Artificial Sequence
  sensor
MSQYDLLETRHALEGIAAYYAALRSTDEDKERIRELHHAIELAQQSGYNSDNVYIMADKQKNGIKANFKIRHNVEDGSVQLADHYQQNTPIGDGPVLLPDNHYLSFQSVLSKDPNEKRDHMVLLEFVTAAGITLGMDELYNVDGGSGGTGSKGEELFTGVVPILVELDGDVNGHKFSVSGEGEGDATYGKLTLKLICTTGKLPVPWPTLVTTLGYGLKCFARYPDHMKQHDFFKSAMPEGYVQERTIFFKDDGNYKTRAEVKFEGDTLVNRIELKGIDFKEDGNILGHKLEYNLDAESNAVLQYQIAVTEAAHNVVLLHLLRCMEPMLAQNVRQNFELLYRSPEMLPLVSSHRTRIFEAIMAGKPEEAREASHRHLAFIEEILLDRSREESRRERSLRRLEQRKN

SEQ ID NO: 28 - protein, 405 aa, Artificial Sequence
  sensor
MSQYDLLETRHALEGIAAYYAALRSTDEDKERIRELHHAIELAQQSGYNSDNVYIMADKQKNGIKANFKIRHNVEDGSVQLADHYQQNTPIGDGPVLLPDNHYLSFQSVLSKDPNEKRDHMVLLEFVTAAGITLGMDELYNVDGGSGGTGSKGEELFTGVVPILVELDGDVNGHKFSVSGEGEGDATYGKLTLKLICTTGKLPVPWPTLVTTLGYGLKCFARYPDHMKQHDFFKSAMPEGYVQERTIFFKDDGNYKTRAEVKFEGDTLVNRIELKGIDFKEDGNILGHKLEYNLDAESNAVLQYQIAVTEAAHNVVLLHLLRCMEPMLAQNVRQNFELLYLVREMLPLVSSHRTRIFEAIMAGKPEEAREASHRHLAFIEEILLDRSREESRRERSLRRLEQRKN

SEQ ID NO: 29 - protein, 405 aa, Artificial Sequence
  sensor
MSQYDLLETRHALEGIAAYYAALRSTDEDKERIRELHHAIELAQQSGYNSDNVYIMADKQKNGIKANFKIRHNVEDGSVQLADHYQQNTPIGDGPVLLPDNHYLSFQSVLSKDPNEKRDHMVLLEFVTAAGITLGMDELYNVDGGSGGTGSKGEELFTGVVPILVELDGDVNGHKFSVSGEGEGDATYGKLTLKLICTTGKLPVPWPTLVTTLGYGLKCFARYPDHMKQHDFFKSAMPEGYVQERTIFFKDDGNYKTRAEVKFEGDTLVNRIELKGIDFKEDGNILGHKLEYNLDAESNAVLQYQIAVTEAAHNVVLLHLLRCMEPMLAQNVRQNFELLYTQEEMLPLVSSHRTRIFEAIMAGKPEEAREASHRHLAFIEEILLDRSREESRRERSLRRLEQRKN

SEQ ID NO: 30 - protein, 405 aa, Artificial Sequence
  sensor
MSQYDLLETRHALEGIAAYYAALRSTDEDKERIRELHHAIELAQQSGYNSDNVYIMADKQKNGIKANFKIRHNVEDGSVQLADHYQQNTPIGDGPVLLPDNHYLSFQSVLSKDPNEKRDHMVLLEFVTAAGITLGMDELYNVDGGSGGTGSKGEELFTGVVPILVELDGDVNGHKFSVSGEGEGDATYGKLTLKLICTTGKLPVPWPTLVTTLGYGLKCFARYPDHMKQHDFFKSAMPEGYVQERTIFFKDDGNYKTRAEVKFEGDTLVNRIELKGIDFKEDGNILGHKLEYNLDAESNAVLQYQIAVTEAAHNVVLLHLLRCMEPMLAQNVRQNFELLYSSTEMLPLVSSHRTRIFEAIMAGKPEEAREASHRHLAFIEEILLDRSREESRRERSLRRLEQRKN

SEQ ID NO: 31 - DNA, 1218 bp, Artificial Sequence
  sensor
atgtcacagtatgacttgctcgaaacacgacacgccctggaaggtatcgccgcttattacgccgcgctgcgtagtaccgatgaagacaaggaacgcatccgtgaactccaccacgccatagagctggcgcagcagtctggcgatctggacgcggaatcaaacgccgtactccagtatcagattgccgtcaccgaagcggcccacaatgtggttctgcttcatctgctaaggtgtatggagccgatgttggcccagaatgtccgccagaacttcgaattgctctattcgcgtcgctacaacagcgacaacgtctatatcatggccgacaagcagaagaacggcatcaaggccaacttcaagatccgccacaacgtcgaggacggcagcgtgcagctcgccgaccactaccagcagaacacccccatcggcgacggccccgtgctgctgcccgacaaccactacctgagcttccagtccgtcctgagcaaagaccccaacgagaagcgcgatcacatggtcctgctggagttcgtgaccgccgccgggatcactctcggcatggacgagctgtacaacgtggatggcggtagcggtggcaccggcagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctcggctacggcctgaagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgtgctgccgctggtgagtagtcaccgcacccgcatatttgaagcgattatggccggtaagccggaagaagcgcgcgaagcatcgcatcgccatctggcctttatcgaagaaattttgctcgacagaagtcgtgaagagagccgccgtgagcgttctctgcgtcgtctggagcaacgaaagaattaa